(19) United States
(12) Patent Application Publication
(10) Pub. No.: US 2024/0115214 A1
(43) Pub. Date: Apr. 11, 2024

(54) MEASUREMENT DEVICE, MEASUREMENT SYSTEM, MEASUREMENT METHOD, AND RECORDING MEDIUM

(71) Applicant: NEC Corporation, Minato-ku, Tokyo (JP)

(72) Inventors: Chenhui HUANG, Tokyo (JP); Zhenwei WANG, Tokyo (JP); Kenichiro FUKUSHI, Tokyo (JP)

(73) Assignee: NEC Corporation, Minato-ku, Tokyo (JP)

(21) Appl. No.: 18/285,308

(22) PCT Filed: Feb. 14, 2022
(86) PCT No.: PCT/JP2022/005593

§ 371 Date: Oct. 2, 2023

(30) Foreign Application Priority Data
Apr. 13, 2021 (JP) 2021-067830

(51) Int. Cl. A61B 5/00; A61B 5/107; A61B 5/11

(52) U.S. Cl. CPC A61B 5/7282; A61B 5/1071; A61B 5/1072; A61B 5/1114; A61B 5/112; A61B 5/1122; A61B 5/486; A61B 5/6807; A61B 2562/0219

(57) ABSTRACT

Provided is a measurement device comprising: a detection unit which detects a walking event from time series data of sensor data pertaining to the movements of feet in order to measure lower limbs on the basis of the sensor data acquired by a single sensor; and a measurement unit which measures the lower limbs by using the sensor data for a prescribed period starting from a walking event timing on the basis of a geometric model to which a constraint condition pertaining to the movements of the lower limbs is given.

### TECHNICAL FIELD

The present disclosure relates to a measurement device or the like that performs measurements of lower limbs.

### BACKGROUND ART

Along with increasing interest in healthcare involving physical condition management, attention has been focused on services for measuring a user's gait including walking characteristics and providing information on the gait to the user. If information related to the lower limbs can be obtained based on data related to gait, it is possible to perform more advanced gait analysis.

NPL 1 discloses a public data set related to dynamics of gait motion of a healthy person. In NPL 1, the dynamics of gait motion is verified based on the trajectories of markers attached to legs, a pelvis, or the like.

PTL 1 discloses a gait analysis system that analyzes the gait state of a walker based on measurement data obtained by sensors attached to legs, a waist, or the like. The system in PTL 1 obtains the angle of the hip joint, knee joints, or ankle joints of a walker using measurement data from sensors attached at positions sandwiching the hip joint, the knee joints, or the ankle joints. The system in PTL 1 obtains the stride length of a walker from measurement data from measurement sensors attached to the dorsum pedis. The system in PTL 1 evaluates the gait state of a walker by comparing a correlation coefficient between the feature point of the joint angle and the stride length with a correlation coefficient obtained in advance between the feature point of angle of a hip joint, knee joints, or ankle joints and a stride length during walking of a healthy person.

PTL 2 discloses an exercise information display system that displays exercise information. The system in PTL 2 generates a moving image in which the motion state of legs of a person who is exercising is reproduced based on values of acceleration and angular velocity measured by a sensor attached to one leg.

### CITATION LIST

### Patent Literatures

PTL 1: Japanese Patent No. 5586050
PTL 2: JP 2016-112108 A

### Non Patent Literature

NPL 1: Fukuchi et al., “A public dataset of overground and treadmill walking kinematics and kinetics in healthy individuals”, (2018), PeerJ, DOI 10.7717/peerj.4640, pp. 1 to 17.

### SUMMARY OF INVENTION

### Technical Problem

According to the methods in NPL 1 and PTL 1, the gait state of a walker is analyzed based on measurement data obtained by sensors attached to a plurality of positions on the legs. That is, according to the methods in NPL 1 and PTL 1, it is necessary to attach sensors to a plurality of positions on the legs of a walker when analyzing the gait of the walker.

According to the method in PTL 2, the length of the lower leg is calculated in accordance with the operation at the time of calibration. According to the method in PTL 2, the positions of the knee and the positions of the bases of the thighs during running are estimated based on the length of the lower leg calculated at the time of calibration. That is, according to the method in PTL 2, the motions of the knee and the bases of the thighs cannot be verified unless the length of the lower leg is measured in advance.

An object of the present disclosure is to provide a measurement device that is capable of performing measurements of lower limbs based on sensor data acquired by a single sensor.

### Solution to Problem

A measurement device according to an aspect of the present disclosure includes a detection unit that detects a gait event from time-series data of sensor data related to motion of a foot, and a measurement unit that performs a measurement of lower limbs by using the sensor data for a prescribed period with a timing of the gait event as a start point, based on a geometric model on which a constraint condition related to motion of the lower limbs is imposed.

In a measurement method according to an aspect of the present disclosure, a computer detects a gait event from time-series data of sensor data related to motion of a foot, and performs a measurement of lower limbs by using the sensor data for a prescribed period with a timing of the gait event as a start point, based on a geometric model on which a constraint condition related to motion of the lower limbs is imposed.

A program according to an aspect of the present disclosure cause a computer to execute detecting a gait event from time-series data of sensor data related to motion of a foot, and perform a measurement of lower limbs by using the sensor data for a prescribed period with a timing of the gait event as a start point, based on a geometric model on which a constraint condition related to motion of the lower limbs is imposed.

### Advantageous Effects of Invention

According to the present disclosure, it is possible to provide a measurement device that is capable of performing measurements of lower limbs based on sensor data acquired by a single sensor.

### BRIEF DESCRIPTION OF DRAWINGS

FIG. 1 is a block diagram illustrating an example of a configuration of a measurement system according to a first example embodiment.

FIG. 2 is a conceptual diagram illustrating an arrangement example of a data acquisition device in the measurement system according to the first example embodiment.

FIG. 3 is a conceptual diagram for describing a coordinate system that is set in the data acquisition device in the measurement system according to the first example embodiment.

FIG. 4 is a conceptual diagram for describing an example of human body planes used in the description of the measurement system according to the first example embodiment.

FIG. 5 is a conceptual diagram for describing an example of a gait cycle used in the description of the measurement system according to the first example embodiment.

FIG. 6 is a block diagram illustrating an example of a configuration of a data acquisition device in the measurement system according to the first example embodiment.

FIG. 7 is a block diagram illustrating an example of a configuration of a measurement device in the measurement system according to the first example embodiment.

FIG. 8 is a conceptual diagram for describing another example of a gait cycle used in the description of the measurement system according to the first example embodiment.

FIG. 9 is a conceptual diagram for describing a method of measuring the distance between the data acquisition device and a heel in a sagittal plane by the measurement device in the measurement system according to the first example embodiment.

FIG. 10 is a conceptual diagram for describing an example of a trajectory of a knee measured for a prescribed period from foot adjacent by the measurement device in the measurement system according to the first example embodiment.

FIG. 11 is a conceptual diagram for describing a third constraint condition imposed on measurement of lower limbs by the measurement device in the measurement system according to the first example embodiment.

FIG. 11 is a conceptual diagram for describing a sixth constraint condition imposed on measurement of lower limbs by the measurement device in the measurement system according to the first example embodiment.

FIG. 13 is a conceptual diagram for describing an example of the trajectory of the knee measured for a period from tibia vertical to heel strike by the measurement device in the measurement system according to the first example embodiment.

FIG. 14 is a conceptual diagram for describing an example of the length of the lower limbs measured by the measurement device in the measurement system according to the first example embodiment.

FIG. 15 is a flowchart for describing an example of the operations of the measurement device in the measurement system according to the first example embodiment.

FIG. 16 is a flowchart for describing an example of first measurement processing by the measurement device in the measurement system according to the first example embodiment.

FIG. 17 is a flowchart for describing an example of second measurement processing by the measurement device in the measurement system according to the first example embodiment.

FIG. 18 is a block diagram illustrating an example of a configuration of a learning system according to a second example embodiment.

FIG. 19 is a conceptual diagram for describing an example of learning by a learning device in the learning system according to the second example embodiment.

FIG. 20 is a block diagram illustrating an example of a configuration of a measurement system according to a third example embodiment.

FIG. 21 is a block diagram illustrating an example of a configuration of a measurement device in the measurement system according to the third example embodiment.

FIG. 22 is a conceptual diagram for describing an example of estimation of physical conditions by the measurement device in the measurement system according to the third example embodiment.

FIG. 23 is a conceptual diagram for describing another example of estimation of physical conditions by the measurement device in the measurement system according to the third example embodiment.

FIG. 24 is a conceptual diagram for describing Application Example 1 according to the third example embodiment.

FIG. 25 is a conceptual diagram for describing Application Example 2 according to the third example embodiment.

FIG. 26 is a conceptual diagram for describing an example of a configuration of a measurement device according to a fourth example embodiment.

FIG. 27 is a conceptual diagram illustrating an example of a hardware configuration for implementing control and processing according to each embodiment.

### EXAMPLE EMBODIMENTS

Hereinafter, example embodiments of the present invention will be described with reference to the drawings. However, the example embodiments described below have technically preferable limitations for carrying out the present invention, but the scope of the invention is not limited to the following. In all the drawings used for describing the following example embodiments, the same reference numerals are given to the same parts unless otherwise specified. In the following example embodiments, repeated description of similar components and operations may be omitted.

### First Example Embodiment

First, a measurement system according to a first example embodiment will be described with reference to the drawings. The measurement system according to the present example embodiment measures sensor data on physical quantities related to motion of a foot by a sensor installed on footwear worn by a user. For example, the physical quantities related to the motion of the foot include acceleration in three axial directions measured by an acceleration sensor (also called spatial acceleration), angular velocity around three axes measured by an angular velocity sensor (also called spatial angular velocity), and the like. The measurement system of the present example embodiment performs measurements of a lower limb based on time-series data in the measured sensor data (also called a gait waveform).

### (Configuration)

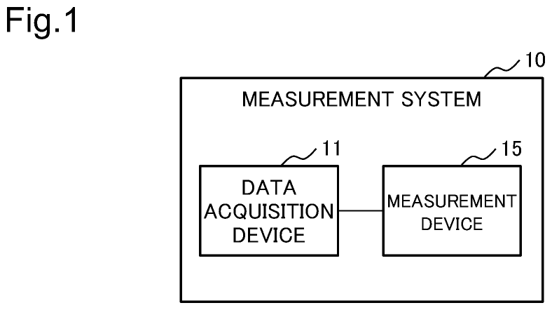

FIG. 1 is a block diagram illustrating an example of a configuration of a measurement system 10 of the present example embodiment. The measurement system 10 includes a data acquisition device 11 and a measurement device 15. The data acquisition device 11 and the measurement device 15 may be connected by wire or wirelessly. The data acquisition device 11 and the measurement device 15 may be configured as a single device. Although only one data acquisition device 11 is illustrated in FIG. 1, one each (two in total) data acquisition device 11 may be arranged on the right and left feet.

The data acquisition device 11 is installed on at least one of the right and left feet. For example, the data acquisition device 11 is installed in footwear such as shoes. In the present example embodiment, the data acquisition device 11 is arranged at positions on the back sides of the arches of the right and left feet as an example. Each data acquisition device 11 includes an acceleration sensor and an angular velocity sensor. The data acquisition device 11 measures, as physical quantities related to the motion of the foot of the user wearing the footwear, physical quantities related to the motion of the foot such as an acceleration in three axis directions (also called spatial acceleration) and an angular velocity around three axes (also called spatial angular velocity). The physical quantities related to the motion of the foot measured by the data acquisition device 11 include not only the acceleration and the angular velocity but also the velocity and the angle calculated by integrating the acceleration and the angular velocity. The physical quantities related to the motion of the foot measured by the data acquisition device 11 also include positions (trajectory) calculated by second-order integration of acceleration. The data acquisition device 11 converts the measured physical quantities into digital data (also called sensor data). The data acquisition device 11 transmits the converted sensor data to the measurement device 15.

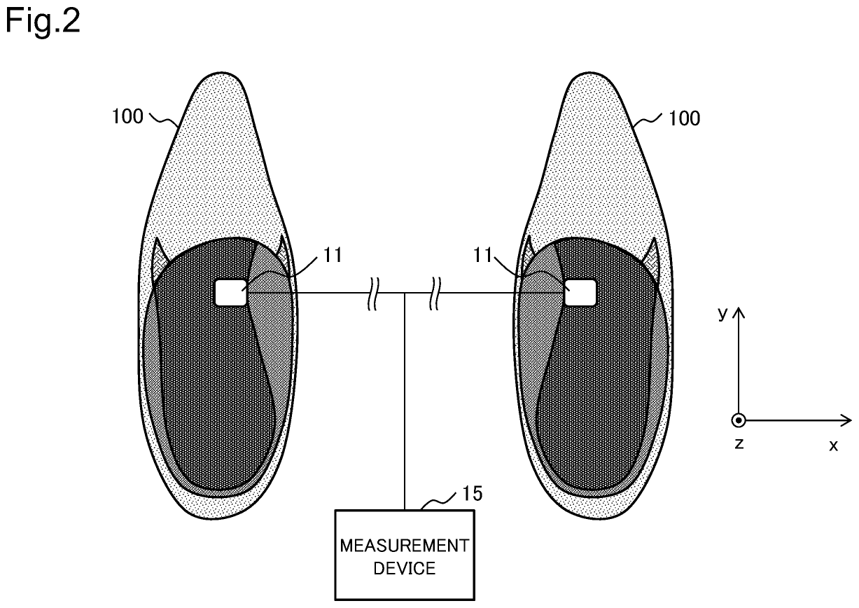

FIG. 2 is a conceptual diagram illustrating an example in which the data acquisition devices 11 are installed in shoes 100. In the example of FIG. 2, the data acquisition devices 11 are installed at positions corresponding to the back sides of the arches of feet. For example, the data acquisition devices 11 are installed in insoles inserted into the shoes 100. For example, the data acquisition devices 11 are installed on the bottom surfaces of the shoes 100. For example, the data acquisition devices 11 are embedded in the main bodies of the shoes 100. The data acquisition devices 11 may be detachable from the shoes 100 or may not be detachable from the shoes 100. The data acquisition devices 11 may be installed at positions that are not on the back sides of the arches of the feet as long as they can acquire sensor data regarding the motion of the feet. The data acquisition devices 11 may be installed on socks worn by the user or decorative articles such as anklets worn by the user. The data acquisition devices 11 may be directly attached to the feet or may be embedded in the feet. FIG. 2 illustrates an example in which the data acquisition device 11 is installed on the shoes 100 of both feet. The data acquisition device 11 may be installed on at least one foot portion. If the data acquisition device 11 is installed on the shoes 100 of both feet, the motion of the feet can be evaluated based on the sensor data measured by the data acquisition devices 11 installed on the right and left feet.

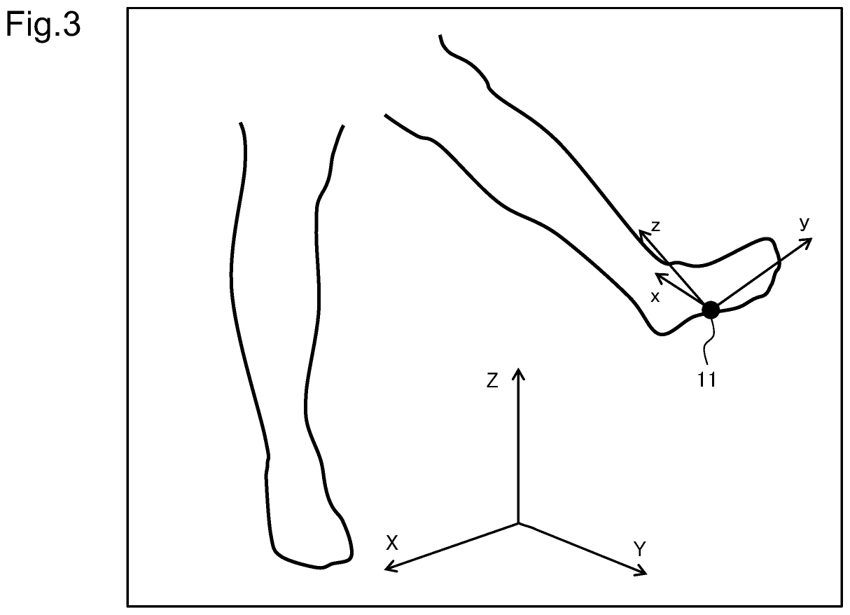

FIG. 3 is a conceptual diagram for describing a local coordinate system (x axis, y axis, z axis) set in the data acquisition device 11 and a world coordinate system (X axis, Y axis, Z axis) set to the ground. In the world coordinate system (X axis, Y axis, Z axis), while the user is standing upright, the lateral direction of the user is set to an X-axis direction (rightward direction is positive), the front direction of the user (traveling direction) is set to a Y-axis direction (forward direction is positive), and a gravity direction is set to a Z-axis direction (vertically upward direction is positive). In the present example embodiment, a local coordinate system is set including an x direction, a y direction, and a z direction with respect to the data acquisition device 11.

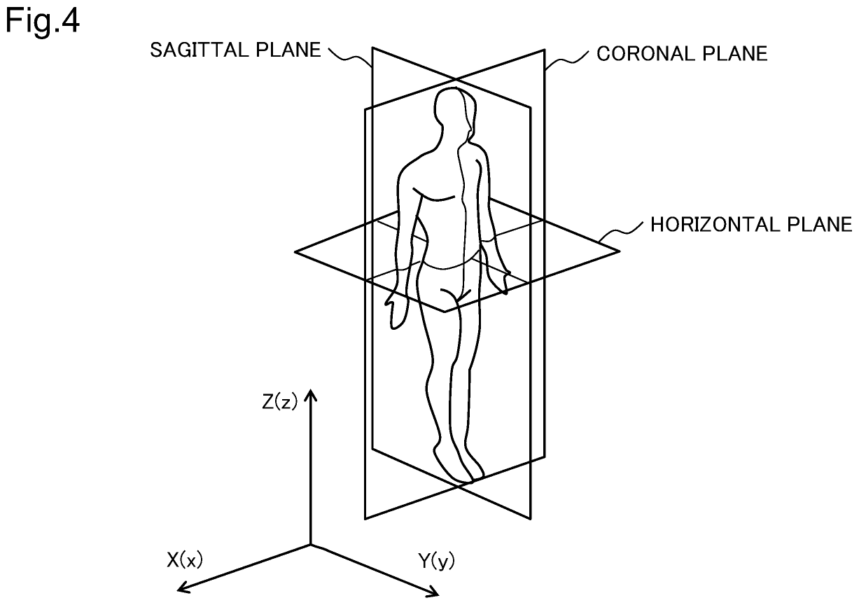

FIG. 4 is a conceptual diagram for describing planes set for a human body (also called human body planes). In the present example embodiment, a sagittal plane dividing the human body into right and left, a coronal plane dividing the body into front and rear, and a horizontal plane dividing the body horizontally are defined. In the upright state as shown in FIG. 4, the world coordinate system coincides with the local coordinate system. In the present example embodiment, rotation in the sagittal plane with the x axis as the rotation axis is defined as roll, rotation in the coronal plane with the y axis as the rotation axis is defined as pitch, and rotation in the horizontal plane with the z axis as the rotation axis is defined as yaw. A rotation angle in the sagittal plane with the x axis as the rotation axis is defined as a roll angle, a rotation angle in the coronal plane with the y axis as the rotation axis is defined as a pitch angle, and a rotation angle in the horizontal plane with the z axis as the rotation axis is defined as a yaw angle. In the present example embodiment, when the human body is viewed from the right side, clockwise rotation in the sagittal plane is defined as positive, and counterclockwise rotation in the sagittal plane is defined as negative. The data acquisition device 11 is implemented by an inertial measurement device including an acceleration sensor and an angular velocity sensor, for example. An example of the inertial measurement device is an inertial measurement unit (IMU). The IMU includes a three-axis acceleration sensor and a three-axis angular velocity sensor. Examples of the inertial measurement device include a vertical gyro (VG) and an attitude heading (AHRS). An example of the inertial device is a global positioning system/inertial navigation system (GPS/INS).

For example, the data acquisition device 11 is connected to the measurement device 15 constructed in a cloud via a mobile terminal (not illustrated) carried by the user. The mobile terminal (not illustrated) is a portable communication device. For example, the mobile terminal is a portable communication device having a communication function, such as a smartphone, a smart watch, or a mobile phone. The mobile terminal receives, from the data acquisition device 11, sensor data on the motion of the user's feet. The mobile terminal transmits the received sensor data to a server or the like on which the measurement device 15 is mounted. The function of the measurement device 15 may be implemented by an application installed in the mobile terminal. In this case, the mobile terminal processes the received sensor data by application software (also called an application) installed in the mobile terminal.

The measurement device 15 acquires sensor data from the data acquisition device 11. The measurement device 15 converts the coordinate system of the acquired sensor data from the local coordinate system to the world coordinate system. The coordinate system of the sensor data may be converted into the world coordinate system by the data acquisition device 11. The measurement device 15 generates time-series data (also called a gait waveform) of the sensor data after conversion into the world coordinate system. The measurement device 15 detects gait events from the gait waveform. Based on the detected gait events, the measurement device 15 performs measurements of the lower limbs using a geometric model on which a constraint condition related to the motion of the lower limbs is imposed. The geometric model is a model for geometrically grasping and verifying the positions, angles, and the like of the parts of the lower limbs with a plurality of timings included in the measurement target period.

For example, the measurement device 15 measures the length of a portion between the knee joint and the ankle joint (also called lower leg) and the length of a portion between the hip joint and the knee joint (also called upper leg). For example, the measurement device 15 measures the positions of the knee joint and the hip joint. For example, the measurement device 15 measures temporal changes (trajectory) in the positions of the knee joint and the hip joint. For example, the measurement device 15 measures the angle of the knee joint.

The detailed method of measurements of the lower limbs by the measurement device 15 will be described later. The measurement device outputs information on the lower limbs. For example, the measurement device 15 outputs information on the lower limbs to a display device (not illustrated) or an external system.

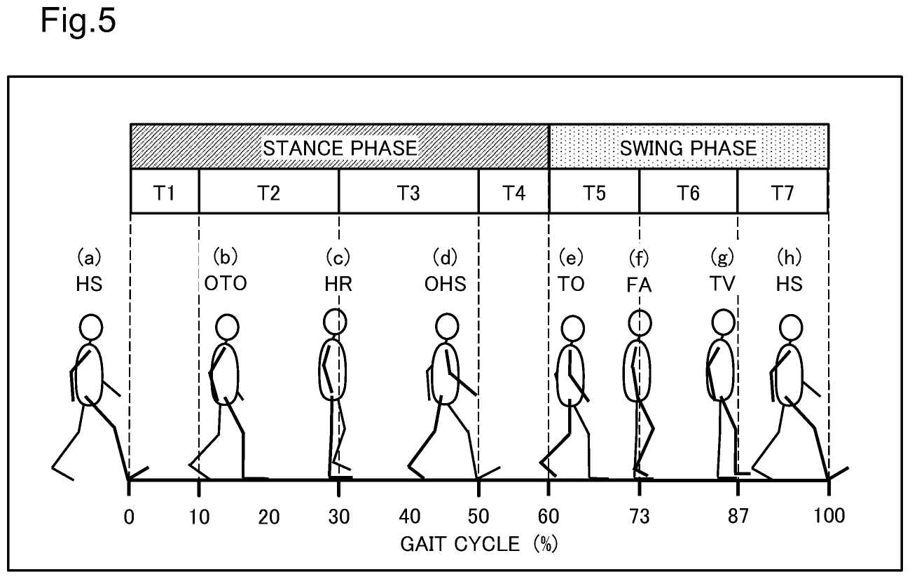

The gait event detected from the gait waveform will be described with reference to the drawings. FIG. 5 is a conceptual diagram for describing one gait cycle with the right foot as a reference. One gait cycle based on the left foot is the same as that of the right foot. The horizontal axis in FIG. 5 indicates a gait cycle that is normalized such that, setting one gait cycle of the right foot as 100%, a time point at which the heel of the right foot lands on the ground is the start point and a time point at which the heel of the right foot next lands on the ground is the end point. The one gait cycle of one foot is roughly divided into a stance phase in which at least a part of the back side of the foot is in contact with the ground and a swing phase in which the back side of the foot is separated from the ground. The stance phase is further subdivided into an initial stance period T1, a mid-stance period T2, a terminal stance period T3, and a pre-swing period T4. The swing phase is further subdivided into an initial swing period T5, a mid-swing period T6, and a terminal swing period T7. Note that FIG. 5 is an example, and does not limit the periods constituting one gait cycle, the names of these periods, and the like.

As illustrated in FIG. 5, in a gait, a plurality of events (also called gait events) occur. FIG. 5(a) illustrates an event in which the heel of the right foot touches the ground (heel strike: HS). FIG. 5(b) illustrates an event in which the toe of the left foot moves away from the ground while the sole of the right foot is grounded (opposite toe off: ONO). FIG. 5(c) illustrates an event in which the heel of the right foot is lifted while the sole of the right foot is grounded (heel rise: HR). FIG. 5(d) illustrates an event in which the heel of the left foot touches the ground (opposite heel strike: OHS). FIG. 5(e) illustrates an event in which the toe of the right foot is separated from the ground while the sole of the left foot is grounded (toe off: TO). FIG. 5(f) illustrates an event in which the left foot and the right foot cross each other while the sole of the left foot is grounded (foot adjacent: FA). FIG. 5(g) illustrates an event in which the tibia of the right foot is substantially vertical to the ground while the sole of the left foot is grounded (tibia vertical: TV). FIG. 5(h) illustrates an event in which the heel of the right foot touches the ground (heel strike: HS). FIG. 5(h) corresponds to the end point of the gait cycle starting from FIG. 5(a) and corresponds to the start point of the next gait cycle. Note that FIG. 5 is an example, and does not limit events that occur during a gait or the names of these events.

### [Data Acquisition Device]

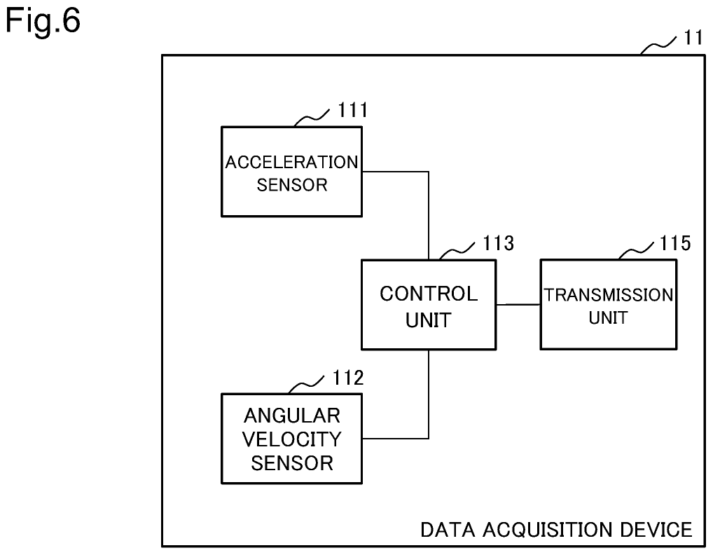

Next, details of the data acquisition device 11 will be described with reference to the drawings. FIG. 6 is a block diagram illustrating an example of a detailed configuration of the data acquisition device 11. The data acquisition device 11 includes an acceleration sensor 111, an angular velocity sensor 112, a control unit 113, and a transmission unit 115. The data acquisition device 11 also includes a power supply (not illustrated).

The acceleration sensor 111 is a sensor that measures acceleration (also called spatial acceleration) in three axial directions. The acceleration sensor 111 outputs the measured acceleration to the control unit 113. For example, the acceleration sensor 111 can be a piezoelectric sensor, a piezoresistive sensor, a capacitance sensor, or the like. The sensor used as the acceleration sensor 111 is not limited in the measurement method as long as the sensor can measure acceleration.

The angular velocity sensor 112 is a sensor that measures an angular velocity (also called a spatial angular velocity) around three axes. The angular velocity sensor 112 outputs the measured angular velocity to the control unit 113. For example, the angular velocity sensor 112 can be a vibration sensor, a capacitance sensor, or the like. The sensor used as the angular velocity sensor 112 is not limited in the measurement method as long as the sensor can measure an angular velocity.

The control unit 113 acquires actual measurement values of acceleration in the three axial directions from the acceleration sensor 111. The control unit 113 acquires an actual measurement value of the angular velocity around the axis from the angular velocity sensor 112. The control unit 113 converts the acquired actual measurement values of the acceleration and the angular velocity into digital data (also called sensor data). The control unit 113 outputs the converted digital data to the transmission unit 115. The sensor data includes at least acceleration data (including acceleration vectors in the three axial directions) and angular velocity data (including angular velocity vectors around the three axes) converted into digital data. The sensor data includes the acquisition times of actual measurement values on which the acceleration data and the angular velocity data are based. The control unit 113 may be configured to output sensor data obtained by making corrections such as implementation error correction, temperature correction, and linearity correction to the acquired acceleration data and angular velocity data. The control unit 113 may convert the coordinate system of the sensor data from the local coordinate system to the world coordinate system. In addition, the control unit 113 may generate angle data (also called plantar angle) around the three axes by using the acquired acceleration data and angular velocity data.

For example, the control unit 113 is a microcomputer or a microcontroller that performs overall control and data processing of the data acquisition device 11. For example, the control unit 113 includes a central processing unit (CPU), a random access memory (RAM), a read only memory (ROM), a flash memory, and the like. The control unit 113 controls the acceleration sensor 111 and the angular velocity sensor 112 to measure the angular velocity and the acceleration. For example, the control unit 113 performs analog-to-digital conversion (AD conversion) on physical quantities (analog data) such as the measured angular velocity and acceleration, and stores the converted digital data in the flash memory. The physical quantities (analog data) measured by the acceleration sensor 111 and the angular velocity sensor 112 may be converted into digital data in each of the acceleration sensor 111 and the angular velocity sensor 112. The digital data stored in the flash memory is output to the transmission unit 115 with a predetermined timing.

The transmission unit 115 acquires sensor data from the control unit 113. The transmission unit 115 transmits the acquired sensor data to the measurement device 15. For example, the transmission unit 115 transmits the sensor data to the measurement device 15 via a wire such as a cable. For example, the transmission unit 115 transmits the sensor data to the measurement device 15 via wireless communication. For example, the transmission unit 115 is configured to transmit sensor data to the measurement device 15 via a wireless communication function (not illustrated) conforming to a standard such as Bluetooth (registered trademark) or WiFi (registered trademark). The communication function of the transmission unit 115 may conform to a standard other than Bluetooth (registered trademark) or WiFi (registered trademark).

### [Measurement Device]

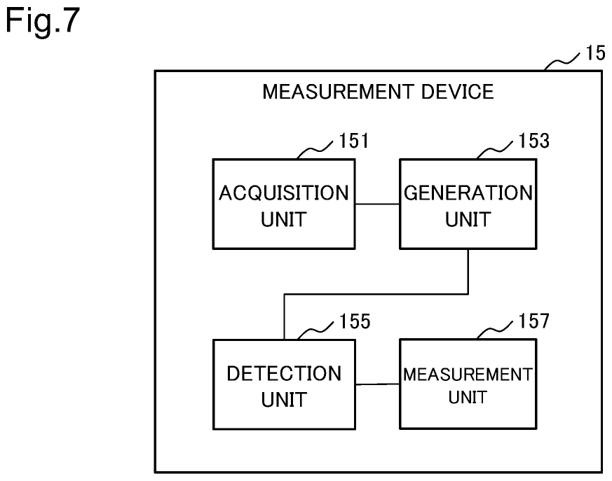

Next, details of the measurement device 15 will be described with reference to the drawings. FIG. 7 is a block diagram illustrating an example of a detailed configuration of the measurement device 15. The measurement device 15 includes an acquisition unit 151, a generation unit 153, a detection unit 155, and a measurement unit 157.

The acquisition unit 151 receives sensor data from the data acquisition device 11. The acquisition unit 151 outputs the received sensor data to the generation unit 153. For example, the acquisition unit 151 receives the sensor data from the data acquisition device 11 via a wire such as a cable. For example, the acquisition unit 151 receives the sensor data from the data acquisition device 11 via wireless communication. For example, the acquisition unit 151 is configured to receive the sensor data from the data acquisition device 11 via a wireless communication function (not illustrated) conforming to a standard such as Bluetooth (registered trademark) or WiFi (registered trademark). The communication function of the acquisition unit 151 may conform to a standard other than Bluetooth (registered trademark) or WiFi (registered trademark).

The generation unit 153 acquires the sensor data from the acquisition unit 151. The generation unit 153 converts the coordinate system of the acquired sensor data from the local coordinate system to the world coordinate system. The generation unit 153 generates time-series data (also called a gait waveform) of the sensor data after conversion into the world coordinate system. The generation unit 153 outputs the generated gait waveform to the detection unit 155.

For example, the generation unit 153 generates a gait waveform of a spatial acceleration or a spatial angular velocity. The generation unit 153 also integrates the spatial acceleration or the spatial angular velocity, and generates a gait waveform of the spatial velocity or the spatial angle (plantar angle). The generation unit 153 also performs second-order integration of the spatial acceleration to generate a gait waveform of the spatial trajectory. The generation unit 153 generates a gait waveform with a predetermined timing or time interval set in accordance with a general gait cycle or a gait cycle unique to the user. The timing with which the generation unit 153 generates a gait waveform can be arbitrarily set. For example, the generation unit 153 is configured to continuously generate a gait waveform during a period in which the gait of the user is continued. The generation unit 153 may also be configured to generate a gait waveform at a specific time.

The detection unit 155 acquires the gait waveform from the generation unit 153. The detection unit 155 detects gait events from the gait waveform. For example, the detection unit 155 detects gait events such as heel strike, toe off, foot adjacent, and tibia vertical. The detection unit 155 outputs the timings of the detected gait events and the values of sensor data for a prescribed period with the timing of a gait event as a start point to the measurement unit 157.

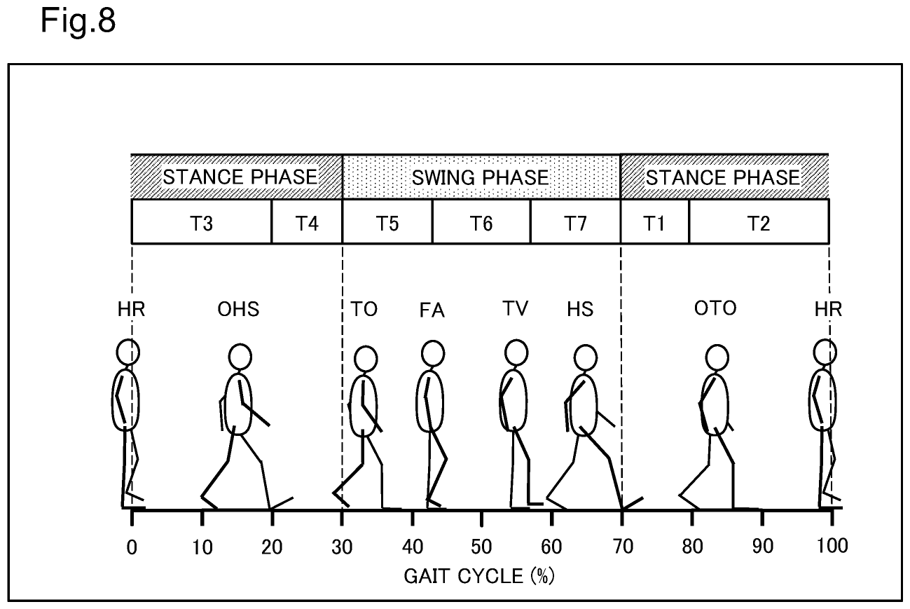

Herein, an example of detection of a gait event by the detection unit 155 will be described. In relation to the present example embodiment, an example will be described in which the timing in the center of the stance phase (the start of the end of terminal stance period T3) is set as the start point of one gait cycle, and the heel strike, the toe off, the foot adjacent, and the tibia vertical are detected as gait events. FIG. 8 is a conceptual diagram for describing an example of one gait cycle with the right foot as a reference, which is set by the detection unit 155. The one gait cycle set by the detection unit 155 starts from the start timing of the terminal stance period T3. The timing of the start of the terminal stance period T3 corresponds to the timing of heel rise. In the following, description will be provided in the order of detection of the gait events, not the order of time series in the gait waveform of one gait cycle.

First, the detection unit 155 cuts out, from the gait waveform at the plantar angle, a gait waveform for one gait cycle starting from the start timing of the terminal stance period T3. Herein, a state in which the toe is positioned above the heel (dorsiflexion) will be defined as negative, and a state in which the toe is positioned below the heel (plantarflexion) will be defined as positive. The timing with which the gait waveform of the plantar angle becomes minimum corresponds to the timing of the start of the stance phase. The timing with which the gait waveform of the plantar angle becomes maximum corresponds to the timing of the start of the swing phase. The timing at a midpoint between the timing of start of the stance phase and the timing of the start of the swing phase corresponds to the timing in the center of the stance phase. The detection unit 155 sets the timing in the center of the stance phase as the start point of one gait cycle. The detection unit 155 also sets the timing in the center of the next stance phase as the end point of one gait cycle.

The detection unit 155 detects, from the gait waveform of the plantar angle in one gait cycle, a timing with which the gait waveform becomes a minimum (first dorsiflexion peak) and a timing with which the gait waveform becomes a maximum (first plantarflexion peak) next to the first dorsiflexion peak. The detection unit 155 further detects, from the gait waveform of the plantar angle in the next one gait cycle, a timing with which the gait waveform becomes a minimum (second dorsiflexion peak) next to the first plantarflexion peak and a timing with which the gait waveform becomes a maximum (second plantarflexion peak) next to the second dorsiflexion peak. The detection unit 155 sets the timing at the midpoint between the timing of the first dorsiflexion peak and the timing of the first plantarflexion peak as the start point of one gait cycle. The detection unit 155 also sets the timing at the midpoint between the timing of the second dorsiflexion peak and the timing of the second plantarflexion peak as the end point of one gait cycle.

The detection unit 155 cuts out a gait waveform in one gait cycle from the gait waveform generated by the generation unit 153. For example, the detection unit 155 cuts out gait waveform data in one gait cycle with the timing at the midpoint between the timing of the first dorsiflexion peak and the timing of the first plantarflexion peak as a start point and with the timing at the midpoint between the timing of the second dorsiflexion peak and the timing of the second plantarflexion peak as an end point. Similarly, the detection unit 155 cuts out a gait waveform in one gait cycle from time-series data of sensor data based on physical quantities (spatial acceleration, spatial angular velocity, and spatial trajectory) related to the motion of the foot measured by the data acquisition device 11.

The detection unit 155 detects the timing of toe off from the gait waveform of the acceleration in the traveling direction (also called Y-direction acceleration). The detection unit 155 detects a maximum peak within a range of 20 to 40% of the gait cycle in the gait waveform of the Y-direction acceleration with the timing of heel rise as a start point. The maximum peak includes two maximum peaks and a minimum peak sandwiched between the maximum peaks. The timing of toe off corresponds to the timing with which the minimum peak sandwiched between the two maximum peaks is detected.

The detection unit 155 detects the timing of heel strike from the gait waveform of the Y-direction acceleration or the vertical acceleration (also called Z-direction acceleration). The detection unit 155 detects the timing of heel strike using a characteristic peak appearing in the vicinity of the timing of heel strike. The detection unit 155 detects a minimum peak around when the gait cycle exceeds 60% at the Y-direction acceleration with the timing of heel rise as a start point. This minimum peak corresponds to the timing of sudden deceleration of the leg in the terminal swing period T7. The detection unit 155 also detects a maximum peak around 70% of the gait cycle during the Y-direction acceleration with the timing of heel rise as a start point. This maximum peak corresponds to the timing of the heel rocker. If the data acquisition device 11 is installed at the position of the arch of the foot, since the data acquisition device 11 is located on the toe side with respect to the rotation axis of the heel joint, the acceleration in the traveling direction (+Y direction) occurs at the time of the motion of the heel rocker (rotation). Therefore, the period of the motion of the heel rocker includes a period during which the acceleration in the gravity direction (Z direction) is converted into the acceleration in the traveling direction (Y direction) by the rotation of the heel rocker along the outer periphery of the grounded heel after heel strike. The timing of heel strike is included for the period from the timing with which the minimum peak is detected to the timing with which the maximum peak is detected. The detection unit 155 detects the timing of the midpoint between the timing with which the minimum peak is detected and the timing with which the maximum peak is detected, as the timing of heel strike. The timing with which the minimum peak is detected at the Y-direction acceleration substantially coincides with the timing with which the maximum peak is detected at the Z-direction acceleration. Therefore, instead of the timing with which the minimum peak is detected at the Y-direction acceleration, the timing with which the maximum peak is detected at the Z-direction acceleration may be used as the timing of sudden deceleration of the foot in the terminal swing period T7.

In the gait waveform of the Z-direction acceleration, the detection unit 155 detects the timing of the maximum peak between toe off and heel strike as the timing of tibia vertical. Tibia vertical is the state where the tibia is approximately vertical to the ground. At the state of tibia vertical, the heel joint is in a neutral state and the plantar surface is vertical to the tibia. That is, in the state of tibia vertical, the roll angle formed by the rotation of the heel joint becomes 0 degree. At the timing with which the roll angle is 0 degree, the peak of the gait waveform at the Z-direction acceleration becomes maximum. That is, tibia vertical corresponds to the timing with the maximum value between toe off and heel strike in the gait waveform at the Z-direction acceleration.

In the gait waveform at the Y-direction acceleration, the detection unit 155 detects, as the timing of foot adjacent, the timing with which the gentle peak on the side close to tibia vertical between tibia vertical and toe off becomes maximum. In the present example embodiment, in a state in which the left foot in contact with the ground is in front of the right foot, the timing in the middle between the timing with which the toe of the right foot passes the position of the heel of the left foot and the timing with which the toe of the right foot passes the position of the toe of the left foot is defined as the timing of foot adjacent.

The measurement unit 157 acquires, from the detection unit 155, the timing of a gait event and the values of the sensor data for a prescribed period with the timing of the gait event as a start point. The measurement unit 157 applies the acquired values of the sensor data to a geometric model on which constraint conditions related to the motion of the lower limbs are imposed, and performs measurements of the lower limbs. For example, the measurement unit 157 measures the length of a portion between the knee joint and the ankle joint (also called lower leg) and the length of a portion between the hip joint and the knee joint (also called upper leg). For example, the measurement unit 157 measures the positions of the knee joint and the hip joint. For example, the measurement unit 157 measures temporal changes (trajectory) in the positions of the knee joint and the hip joint. For example, the measurement unit 157 measures the angle of the knee joint.

### [Measurement Processing]

Next, measurement processing by the measurement unit 157 will be described with an example. The measurement processing by the measurement unit 157 includes first measurement processing and second measurement processing. The first measurement processing is a process of measuring the trajectory of the knee joint (knee) for a prescribed period with foot adjacent as a start point. The second measurement processing is a process of measuring the trajectory of the hip joint (pelvis) and the angle of the knee joint for the period from tibia vertical to heel strike.

### <First Measurement Processing>

Next, the first measurement processing will be described with reference to the drawings. In the first measurement processing, the measurement unit 157 measures the trajectory of the knee in the mid-swing period T6 of the swing phase, using the value of the sensor data for the prescribed period with foot adjacent as a start point.

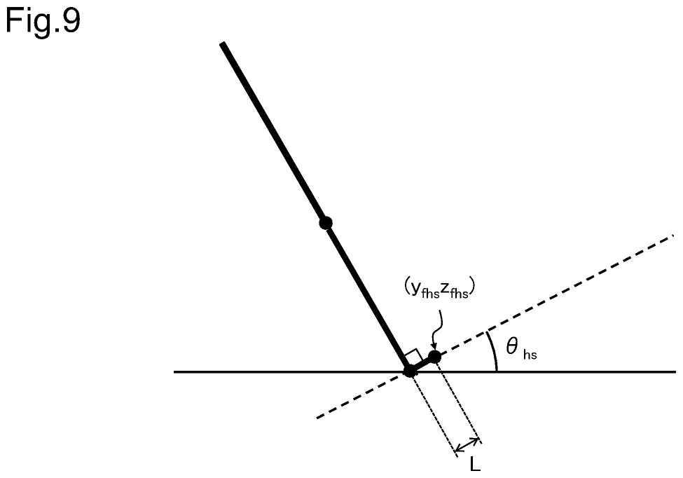

The measurement unit 157 calculates a distance L between the data acquisition device 11 and the ankle joint, using the value of the plantar angle at the timing of heel strike. FIG. 9 is a conceptual diagram for describing a method of measuring the distance L between the data acquisition device 11 and the ankle joint in a sagittal plane. At the timing of heel strike, it is assumed that the angle formed by the lower leg and the planar surface is a right angle. For example, the measurement unit 157 calculates the distance L between the data acquisition device 11 and the ankle joint by substituting a position (y_{fhs}, z_{fhs}) of the data acquisition device 11 and a plantar angle θₕₛ at the timing of heel strike into the following equation 1 or equation 2.

L=y_{fhs}/cos θₕₛ (1)

L=z_{fhs}/sin θₕₛ (2)

For example, the measurement unit 157 may use an average value of the distances L calculated using the above equations 1 and 2. For example, the measurement unit 157 measures the distance L between the data acquisition device 11 and the ankle joint in each gait cycle. For example, the measurement unit 157 may measure the distance L between the data acquisition device 11 and the ankle joint immediately after activation or at the timing of calibration. For example, the distance L between the data acquisition device 11 and the ankle joint may be measured in advance and stored in a storage unit (not illustrated) accessible from the measurement unit 157.

Next, the measurement unit 157 measures the trajectory of the knee in the mid-swing period T6 and the terminal swing period T7 using the value of the sensor data for the period from the timing of foot adjacent to heel strike. In the mid-swing period T6 and the terminal swing period T7, the measurement unit 157 measures the trajectory of the knee under the following first to third constraint conditions. The first constraint condition is based on the knowledge of biomechanics disclosed in NPL 1 (NPL 1: Fukuchi et al., “A public dataset of overground and treadmill walking kinematics and kinetics in healthy individuals”, 2018, PeerJ, DOI 10.7717/peerj. 4640, pp. 1 to 17).

The first constraint condition is a condition that “the angle formed by the lower leg (tibia) and the planar surface is a right angle for the period from foot adjacent to heel strike”. In other words, the ankle joint is almost neutral (≈90 degrees) for the period from foot adjacent to heel strike (the mid-swing period T6 to the terminal swing period T7). FIG. 3J (Ankle Dorsi/plantarflexion) on page 8 of NPL 1 discloses data indicating that the ankle joint is almost neutral (within ±5 degrees) from the mid-swing period T6 to the terminal swing period T7. In the present example embodiment, the angle formed by the lower leg (tibia) and the planar surface is regarded as a right angle for the period from foot adjacent to heel strike.

The second constraint condition is a condition that “the extension/flexion of the knee joint is a rotational motion around the knee joint”. In the present example embodiment, the motion of the knee joint is verified in a polar coordinate system centered on the knee joint. In the case of three-dimensional analysis, the trajectory of the knee joint may be verified in a spherical coordinate system.

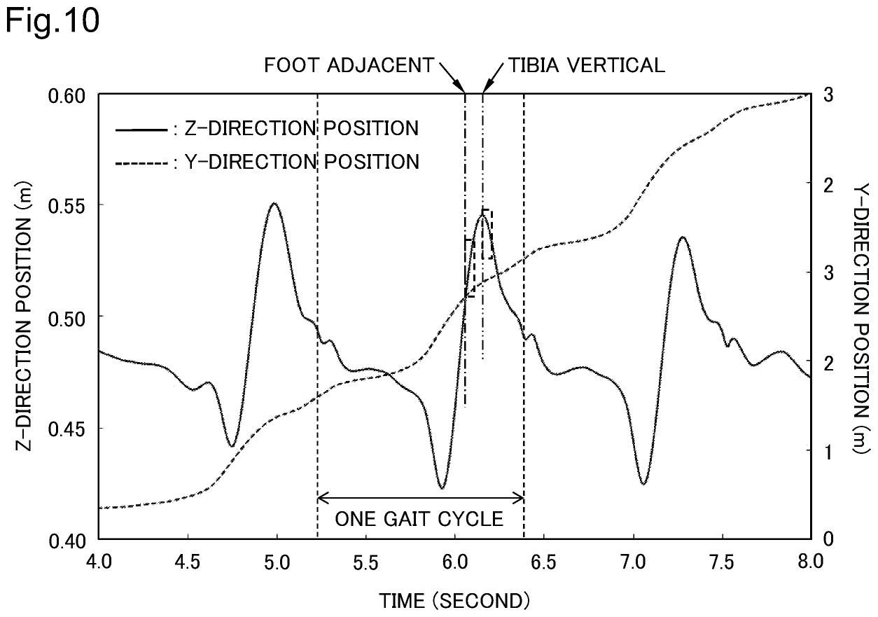

The third constraint condition is a condition that “the knee performs isokinetic motion for a prescribed time (measurement target time frame) with foot adjacent/tibia vertical as a start point”. FIG. 10 is a graph showing an example of temporal changes (trajectory) in the position of the knee measured in accordance with the gait of a person wearing e-skin (registered trademark) manufactured by Xenoma Inc. (registered trademark). For example, the trajectory of the knee is measured by applying the motion of each part of the body to a skeleton/muscle model. FIG. 10 includes a trajectory (solid line) of the Z-direction position of the knee and a trajectory (broken line) of the Y-direction position of the knee. In the present example embodiment, it is assumed that the knee performs a constant velocity motion for the period immediately after foot adjacent (within the range of the frame indicated by the alternate long and short dash line in FIG. 10) and for the period immediately after tibia vertical (within the range of the frame indicated by the alternate long and two short dashes line in FIG. 10).

The measurement unit 157 sets a first relative coordinate system (also called knee origin coordinate system at the time of foot adjacent) in which the position of the knee joint at the timing of foot adjacent is set as an origin. The measurement unit 157 measures the trajectory of the knee joint in the first relative coordinate system under the first to third constraint conditions. The measurement unit 157 converts the measured trajectory of the knee joint in a first relative coordinate system into the world coordinate system. In the present example embodiment, the origin of the world coordinate system in the first relative coordinate system is expressed as (y′₀, z′₀).

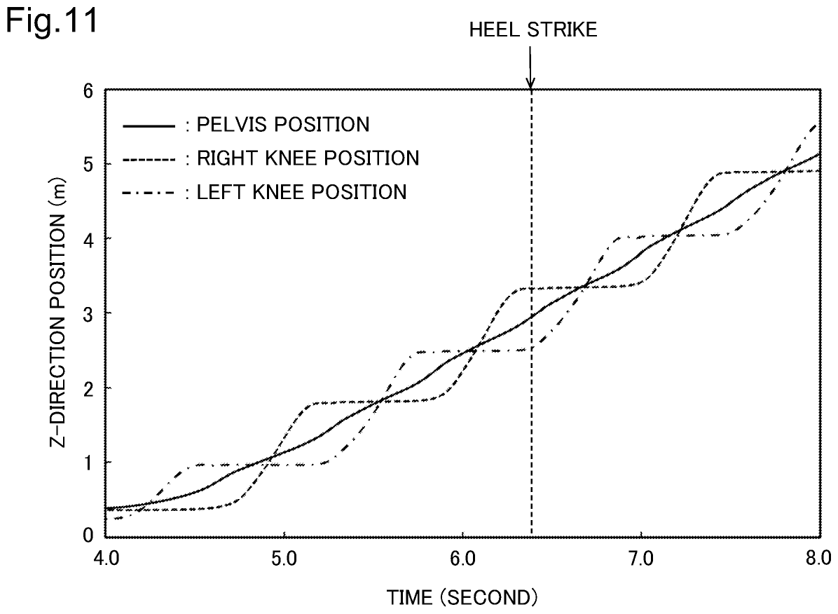

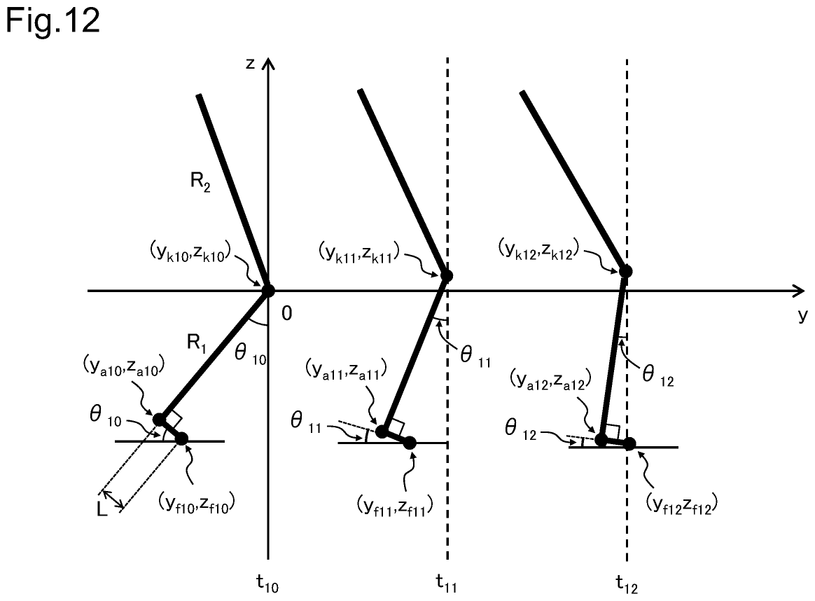

FIG. 11 is a conceptual diagram for describing measurement of trajectory of the knee immediately after foot adjacent (mid-swing period T6). FIG. 11 illustrates the state of the leg at timing t₁₀ of foot adjacent, timing t₁₁ and timing t₁₂ included for a prescribed period from the timing t₁₀ of foot adjacent. Hereinafter, symbols of the timings t₁₀ to t₁₂ will described as t₁ᵢ (i=0, 1, 2). The measurement value from the data acquisition device 11 converted into the first relative coordinate system is expressed as (Yᵢ, Zᵢ). FIG. 11 illustrates an example in which the values of sensor data with the three timings are used. Alternatively, values of sensor data with four or more timings may be used. The measurement unit 157 calculates the trajectory of the knee for the period from the timing of foot adjacent to the timing of heel strike by the following procedure (the mid-swing period T6 and the terminal swing period T7).

The measurement unit 157 converts the positions of the knee joint and the heel into the polar coordinate system based on the second constraint condition. The position (yₐ₁ᵢ, zₐ₁ᵢ) of the heel in the polar coordinate system at timing t₁ᵢ have relationships in the following equations 3 and 4.

yₐ₁ᵢ=y_{f1i}−L cos θ₁ᵢ (3)

zₐ₁ᵢ=z_{f1i}−L sin θ₁ᵢ (4)

In the equations 3 and 4, (y_{f1i}, z_{f1i}) is the position of the polar coordinate system data acquisition device 11 at the timing t₁ᵢ, and θ₁ᵢ is the plantar angle at the timing t₁ᵢ.

The position (y_{f1i}, z_{f1i}) of the data acquisition device 11 at the timing t₁ᵢ has relationships in the following equations 5 and 6.

y_{f1i}=y′₀+Yᵢ (5)

z_{f1i}=z′₀+Zᵢ (6)

In the equations 5 and 6, (Yᵢ, Zᵢ) is the position of the data acquisition device 11 in the first relative coordinate system at the timing 1i.

When the equation 3 is substituted into the equation 5 and the equation 4 is substituted into the equation 6 based on the third constraint condition, relationships in the following equations 7 and 8 are obtained.

yₐ₁ᵢ=R₁ sin θ₁ᵢ+v_{ky}(Tᵢ−T₀)=y′₀+Yᵢ−L cos θ₁ᵢ (7)

zₐ₁ᵢ=R₁ cos θ₁ᵢ+v_{kz}(Tᵢ−T₀)=z′₀+Zᵢ−L sin θ₁ᵢ (8)

In the equations 7 and 8 above, v_{ky} is the knee speed in the Y direction at the timing t₁₀ to t₁₂, and v_{kz} is the knee speed in the Z direction at the timing t₁₀ to t₁₂.

When the equations 5 to 8 at the timing t₁₀ and the timing t₁₂ are used, relationships in the following equations 9 and 10 are obtained.

R₁ sin θ₁₁+v_{ky}(T₁−T₀)−R₁ sin θ₁₀=Y₁−Y₀+L(cos θ₁₀−cos θ₁₁) (9)

R₁ cos θ₁₁+v_{kz}(T₁−T₀)−R₁ cos θ₁₀=Z₁−Z₀+L(sin θ₁₀−sin θ₁₁) (10)

Similarly, when Equations 5 to 8 at the timing t₁₁ and the timing t₁₂ are used, relationships in the following equations 11 and 12 are obtained.

R₁ sin θ₁₂+v_{ky}(T₂−T₁)−R₁ sin θ₁₁=Y₂−Y₁+L(cos θ₁₁−cos θ₁₂) (11)

R₁ cos θ₁₂+v_{kz}(T₂−T₁)−R₁ cos θ₁₁=Z₂−Z₁+L(sin θ₁₁−sin θ₁₂) (12)

The measurement unit 157 substitutes the value θ₁ᵢ of the plantar angle at the timing t₁ᵢ into the above the equations 9 to 12, and calculates v_{kz} with a length R₁ of the thigh, the speed v_{ky} of the knee in the Y direction, and the speed of the knee in the Z direction.

The measurement unit 157 calculates a knee position (Yₖ₁ᵢ, Zₖ₁ᵢ) in the world coordinate system at the timing t₁ᵢ as in the following equations 13 and 14.

Yₖ₁ᵢ=Y₀−L cos θ₁ᵢ+R₁ sin θ₁ᵢ (13)

Zₖ₁ᵢ=Z₀−L sin θ₁ᵢ+R₁ cos θ₁ᵢ (14)

The measurement unit 157 calculates the position (Yₖ₁ᵢ, Zₖ₁ᵢ) of the knee in the world coordinate system for the period from the timing of foot adjacent to the timing of heel strike by substituting the plantar angle θ₁ᵢ at the timing t₁ᵢ into the above the equations 13 and 14. When the positions of the knee for the prescribed period with foot adjacent as a start point are connected in time series, the trajectory of the knee is obtained.

### <Second Measurement Processing>

Next, the second measurement processing will be described with reference to the drawings. In the second measurement processing, the measurement unit 157 measures the trajectory of the pelvis and the knee joint angle in the terminal swing period T7 by using the values of the sensor data for the period from tibia vertical to heel strike. In the present example embodiment, the position of the hip joint in the sagittal plane is regarded as the position of the pelvis. The rotation of the sole of the foot during the period near tibia vertical is considered to be due to the motion of the thigh and lower leg. Herein, the rotation of the sole of the foot is verified by the angle of the sole of the foot (plantar angle) with respect to the ground in the sagittal plane. In the terminal swing period T7, the trajectory of the knee is measured under the following fourth to sixth constraint conditions. The fourth constraint condition and the fifth constraint condition are based on the knowledge of biomechanics disclosed in NPL 1.

The fourth constraint condition is a condition that “the hip joint angle is constant for the period from tibia vertical to heel strike”. FIG. 3D (Hip Flexion/Extension) on page 8 of NPL 1 discloses data indicating that the hip joint angle is substantially constant from the latter half of the mid-swing period T6 to the terminal swing period T7. In the present example embodiment, it is considered that the hip joint angle is fixed for the period from tibia vertical to heel strike.

The fifth constraint condition is a condition that “the thigh and the lower leg are in a straight line immediately before heel strike”. FIG. 3G (Knee Fix/Extension) on page 8 of NPL 1 discloses data indicating that the thigh and the lower thigh are substantially in a straight line immediately before heel strike and the knee joint is substantially in an extended state. In the present example embodiment, it is assumed that the thigh and the lower leg are in a straight line at the timing of heel strike.

The sixth constraint condition is a condition that “the position of the pelvis in the sagittal plane at the timing of heel strike is the position midway between the both knees”. FIG. 12 is a graph illustrating an example of temporal changes (trajectory) in the positions of the right and left knees and the pelvis in the traveling direction (Y direction) in the sagittal plane, measured by motion capture. The distance between the right and left knees in the sagittal plane is maximized at the timing of heel strike. At the timing of heel strike, the position of the pelvis in the traveling direction (Y direction) in the sagittal plane corresponds to the position midway between the right and left heels in the traveling direction (Y direction) in the sagittal plane.

Under the fourth to sixth constraint conditions, the measurement unit 157 calculates the trajectory of the hip joint in the second relative coordinate system with the position of the knee joint at the time point of tibia vertical as the origin (tibia vertical knee joint origin coordinate system).

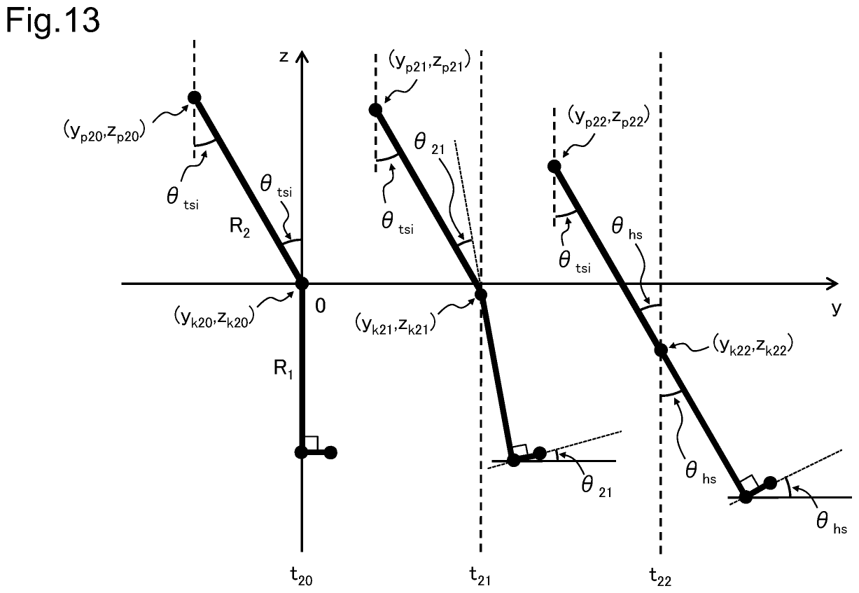

FIG. 13 is a conceptual diagram for describing measurement of the trajectory of the pelvis in the terminal swing period T7. FIG. 13 illustrates the state of the leg at timing t₂₀ of tibia vertical, timing t₂₁ included for a prescribed period with the timing t₂₀ of tibia vertical as a start point, and timing t₂₂ of heel strike. Hereinafter, symbols of the timings t₂₀ to t₂₂ will be described as t₂ᵢ (i=0, 1, 2). FIG. 13 illustrates an example in which the values of sensor data with the three timings are used. Alternatively, values of sensor data with four or more timings may be used. The measurement unit 157 calculates the trajectory of the pelvis in the terminal swing period T7 by the following procedure.

The measurement unit 157 calculates a knee joint angle θₜₛᵢ under the fourth and fifth constraint conditions. Under the fourth and fifth constraint conditions, the angle of the thigh with respect to the normal line (Z axis) of the ground is constant in the terminal swing period T7. The measurement unit 157 calculates the knee joint angle θₜₛᵢ at timing t₂ᵢ using the following equation 15.

θₜₛᵢ=θₕₛ−θ₂ᵢ (15)

A plantar angle θ₂₀ at the timing t₂₀ of tibia vertical is 0, and a plantar angle θ₂₂ at the timing t₂₂ of heel strike is θₕₛ.

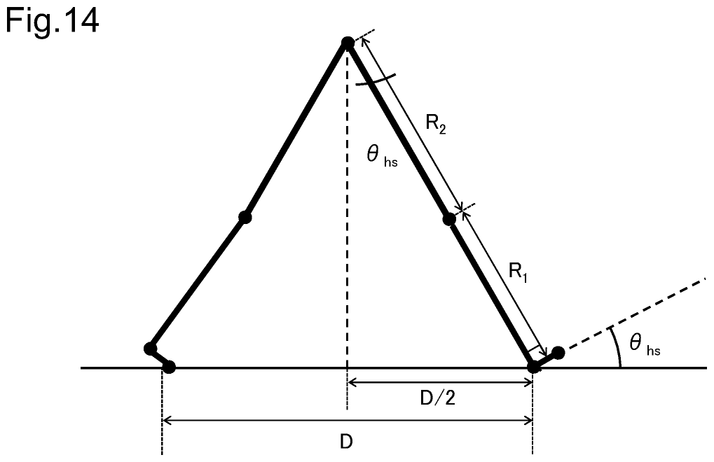

The measurement unit 157 calculates a length R₂ of the thigh under the sixth constraint condition. FIG. 14 is a conceptual diagram for describing a method of calculating the length R₂ of the thigh. The measurement unit 157 uses the sensor data to calculate a stride length D. The stride length D corresponds to the movement distance of the data acquisition device 11 in the Y direction at the timing of continuous heel strike, continuous toe off, or the like. The measurement unit 157 calculates the length R₂ of the thigh using the following equation 16.

$${R}_{2} = \frac{D}{2} \times sin {θ}_{h s} - {R}_{1} \qquad ( 16 )$$

For example, the measurement unit 157 uses the stride length calculated by the following procedure.

For example, the measurement unit 157 measures, as the stride length, the movement distance of the data acquisition device 11 in the Y direction at the timing of continuous heel strike, continuous toe off, or the like. The movement distance of the data acquisition device 11 in the Y direction can be calculated based on a trajectory calculated by performing second-order integration of the Y-direction acceleration. For example, the difference between the positions in the Y direction in the continuous heel strike or continuous toe off corresponds to the stride length. The measurement unit 157 may calculate, as the stride length, the difference between the positions in the Y direction for a period of a continuous arbitrary gait event without being limited to heel strike and toe off.

For example, the measurement unit 157 may measure the stride length based on the timings of toe off, heel strike, and foot adjacent. The measurement unit 157 extracts a section between toe off and heel strike, as a gait waveform of a Y-direction trajectory of one step, from the gait waveform of the Y-direction trajectory. The measurement unit 157 calculates the absolute value of the difference between the spatial position at foot adjacent and the spatial position at toe off, using the gait waveform of the Y-direction trajectory of one step. The absolute value of the difference between the spatial position at foot adjacent and the spatial position at toe off corresponds to a left foot step length (also called a first step length) in a state where the left foot is in the front and the right foot is in the back. The measurement unit 157 also calculates the absolute value of the difference between the spatial position at the timing of foot adjacent and the spatial position at heel strike, using the gait waveform of the Y-direction trajectory of one step. The absolute value of the difference between the spatial position at the timing of foot adjacent and the spatial position at heel strike corresponds to a right foot step length (also called a second step length) in a state where the right foot is in front and the left foot is in back. The sum of the right foot step length and the left foot step length corresponds to the stride length. According to this method, the step length of each foot can be individually measured.

The measurement unit 157 calculates a position (yₚ₂ᵢ, zₚ₂₁) of the pelvis in the second relative coordinate system at the timing t₂ᵢ using the following equations 17 and 18.

yₚ₂ᵢ=yₖ₂ᵢ+R₂ COS θₕₛ (17)

zₚ₂ᵢ=zₖ₂ᵢ+R₂ sin θₕₛ (18)

The measurement unit 157 substitutes a position of the knee (yₖ₂₁, zₖ₂₁) into each of the equations 17 and 18 to calculate the position of the pelvis (yₚ₂ᵢ, zₚ₂₁) in the second relative coordinate system. The position of the knee (yₖ₂₁, zₖ₂₁) is measured by the method of the first measurement processing.

For example, the measurement unit 157 converts the coordinate system of the position of the pelvis in the second relative coordinate system from the second relative coordinate system (yₚ₂ᵢ, zₚ₂₁) to the world coordinate system (Yₚ₂i, Zₚ₂₁) by using the following equations 19 and 20.

Yₚ₂ᵢ=yₚ₂ᵢ+yₖ₂₀ (19)

Zₚ₂ᵢ=z_{pzi}+zₖ₂₀ (20)

In the above equations 19 and 20, (yₖ₂₀, zₖ₂₀) indicates the position of the knee in the world coordinate system at timing t₂₀ of tibia vertical.

In the case of measuring the three-dimensional position of the pelvis, a spherical coordinate system may be used instead of a polar coordinate system, including the length of the pelvis in the horizontal direction (X direction). For example, in the case of performing three-dimensional measurement, a constraint condition that the velocity is constant in the x direction is imposed, and the position of the pelvis is measured using a determinant for conversion from a polar coordinate system to a spherical coordinate system. According to the three-dimensional measurement, the motion of the pelvis can be verified three-dimensionally.

The measurement unit 157 outputs information on the measured motion of the lower limbs. For example, the measurement unit 157 outputs information on the trajectory of the knee joint in the mid-swing period T6 and the terminal swing period T7 and information on the trajectory of the knee joint and the pelvis (hip joint) in the terminal swing period T7. For example, the measurement unit 157 outputs information on the motion of the lower limbs to a display device (not illustrated). The information on the motion of the lower limbs output to the display device is displayed on the screen of the display device. For example, the measurement unit 157 outputs information on the motion of the lower limbs to an external system. The information on the motion of the lower limbs output to the external system is used for an arbitrary purpose.

As described above, the measurement unit 157 calculates the trajectory of the knee joint based on the geometric model in the first relative coordinate system (the foot adjacent origin coordinate system) with the position of the knee joint at the timing of foot adjacent as the origin. The measurement unit 157 calculates the trajectory of the hip joint (pelvis) and the angle of the knee joint, based on the geometric model in the second relative coordinate system (tibia vertical origin coordinate system) with the position of the knee joint at the time point of tibia vertical as the origin.

For example, if a length R1 of the lower leg is known, the calculation can be simplified. For example, the calculation can be simplified by calculating the length R1 of the lower leg for several steps at the start of a gait, and then using a calculated value of the length R1 of the lower leg. For example, in making initial setting and calibration for using the measurement device 15, the length R1 of the lower leg, a length R2 of the upper leg, and the distance L between the data acquisition device 11 and the ankle joint are obtained, and these values are recorded in a storage unit (not illustrated). In the measurement regarding the lower limbs, the calculation can be simplified by using the length R1 of the lower leg, the length R2 of the upper leg, and the distance L between the data acquisition device 11 and the ankle joint recorded in the storage unit.

### (Operations)

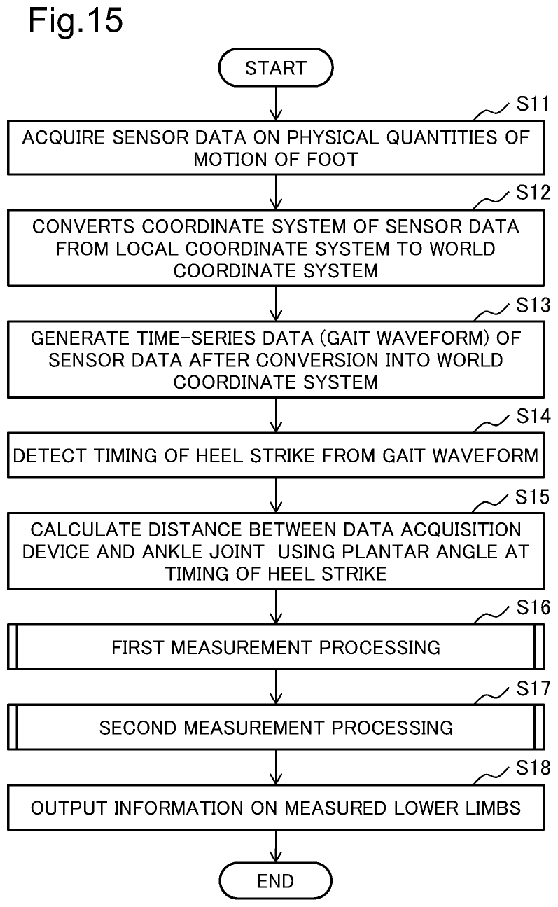

Next, the operations of the measurement device 15 in the measurement system 10 of the present example embodiment will be described with reference to the drawings. In the following description, the measurement device 15 is described as mainly performing the operations. FIG. 15 is a flowchart for describing an example of the operations of the measurement device 15.

Referring to FIG. 15, first, the measurement device 15 acquires, from the data acquisition device 11, sensor data on the physical quantities of the motion of the foot of a walker walking in footwear on which the data acquisition device 11 is installed (step S11). The measurement device 15 acquires sensor data in a local coordinate system set in the data acquisition device 11. For example, the measurement device 15 acquires data of spatial acceleration and spatial angular velocity as the sensor data on the motion of the foot.

The measurement device 15 then converts the coordinate system of the sensor data from the local coordinate system of the data acquisition device 11 to the world coordinate system (step S12).

The measurement device 15 then generates time-series data (gait waveform) of the sensor data after the conversion into the world coordinate system (step S13). For example, the measurement device 15 generates a gait waveform at accelerations in the X direction, the Y direction, and the Z direction. For example, the measurement device 15 generates a gait waveform at an angular velocity around the X axis, the Y axis, and the Z axis. For example, the measurement device 15 generates a gait waveform at a spatial angle (plantar angle) by using sensor data of at least one of the spatial acceleration and the spatial angular velocity. For example, the measurement device 15 generates time-series data of the spatial velocity and the spatial trajectory.

The measurement device 15 then detects the timing of heel strike from the gait waveform (step S14). For example, the measurement device 15 detects the timing of heel strike from the gait waveform at the Y-direction acceleration and the Z-direction acceleration.

The measurement device 15 then calculates the distance between the data acquisition device 11 and the ankle joint using the plantar angle at the timing of heel strike (step S15).

The measurement device 15 then executes first measurement processing (step S16). In the first measurement processing, the measurement device 15 measures the trajectory of the knee joint for a prescribed period with foot adjacent as a start point under the first to third constraint conditions. For example, the prescribed period is a period immediately after foot adjacent. For example, the prescribed period is a period from foot adjacent to tibia vertical. For example, the prescribed period is a period from to foot adjacent to heel strike.

The measurement device 15 executes second measurement processing (step S17). In the second measurement processing, the measurement device 15 measures the trajectory of the knee joint or the pelvis (hip joint) and the angle of the knee joint for the period from tibia vertical to heel strike (terminal swing period T7) under the fourth to sixth constraint conditions.

The measurement device 15 outputs information on the measured lower limbs (step S18). For example, the information on the lower limbs output from the measurement device 15 is output to a display device (not illustrated) or an external system.

### [First Measurement Processing]

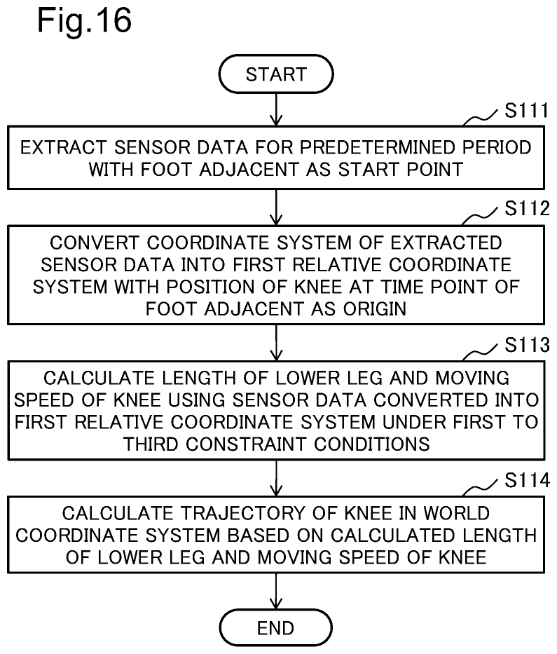

Next, details of the first measurement processing (step S16 in FIG. 15) by the measurement device 15 will be described with reference to the drawings. FIG. 16 is a flowchart for describing an example of first measurement processing by the measurement device 15. In the description with reference to the flowchart in FIG. 16, the measurement device 15 is described as mainly performing the operations.

Referring to FIG. 16, first, the measurement device 15 extracts sensor data for a prescribed period with foot adjacent as a start point (step S111).

The measurement device 15 then converts the coordinate system of the extracted sensor data into the first relative coordinate system with the position of the knee (knee joint) at the timing of foot adjacent as the origin (step S112).

The measurement device 15 then calculates the length of the lower leg and the moving speed of the knee using the sensor data converted into the first relative coordinate system under the first to third constraint conditions (step S113).

The measurement device 15 then calculates the trajectory of the knee in the world coordinate system based on the calculated length of the lower leg and the calculated moving speed of the knee (step S114).

### [Second Measurement Processing]

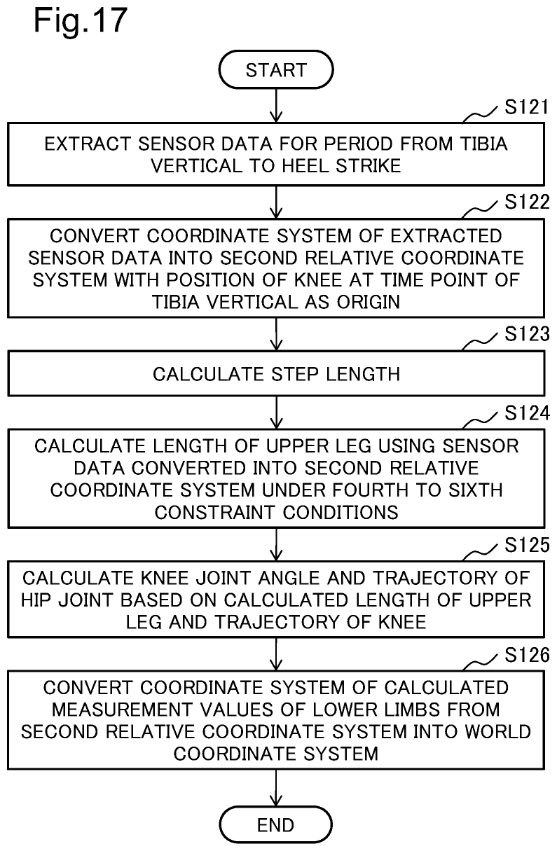

Next, details of the second measurement processing (step S17 in FIG. 15) by the measurement device 15 will be described with reference to the drawings. FIG. 17 is a flowchart for describing an example of second measurement processing by the measurement device 15. In the description with reference to the flowchart in FIG. 17, the measurement device 15 is described as mainly performing the operations.

Referring to FIG. 17, first, the measurement device 15 extracts sensor data for a period from tibia vertical to heel strike (step S121).

Next, the measurement device 15 converts the coordinate system of the extracted sensor data into the second relative coordinate system with the position of the knee (knee joint) at the time point of tibia vertical as the origin (step S122).

The measurement device 15 then calculates a step length (step S123). For example, the measurement device 15 measures, as the stride length, the movement distance of the data acquisition device 11 in the Y direction at the timing of continuous heel strike, continuous toe off, or the like. For example, the measurement device 15 measures the stride length based on the timings of toe off, heel strike, and foot adjacent. The measurement of the stride length may take on a value measured in advance.

The measurement device 15 then calculates the length of the upper leg using the sensor data converted into the second relative coordinate system under the fourth to sixth constraint conditions (step S124).

The measurement device 15 then calculates the knee joint angle and the trajectory of the hip joint (pelvis) based on the calculated length of the state and the trajectory of the knee (step S125).

The measurement device 15 then converts the coordinate system of the calculated measurement values of the lower limbs from the second relative coordinate system into the world coordinate system (step S126).

As described above, the measurement system of the present example embodiment includes the data acquisition device and the measurement device. The data acquisition device is arranged on a user's footwear. The data acquisition device measures a spatial acceleration and a spatial angular velocity according to the gait of the user. The data acquisition device generates sensor data based on the measured spatial acceleration and spatial angular velocity. The data acquisition device outputs the generated sensor data to the measurement device. The measurement device includes the acquisition unit, the generation unit, the detection unit, and the measurement unit. The acquisition unit acquires sensor data related to the motion of the foot. The generation unit generates time-series data of sensor data related to the motion of the foot. The detection unit detects gait events from the time-series data of sensor data related to the motion of the foot. The measurement unit performs measurements of the lower limbs based on a geometric model on which constraint conditions related to the motion of the lower limbs are imposed, using sensor data for a prescribed period with the timing of a gait event as a start point.

The measurement device of the present example embodiment performs measurements of the lower limbs (upper legs/lower legs) using time-series data of sensor data acquired by a single data acquisition device (sensor) according to a natural gait motion. The measurement device of the present example embodiment performs measurements of the lower limbs based on knowledge of biomechanics. For example, the measurement device of the present example embodiment measures the trajectory of the knee and the pelvis (hip joint) and the angle of the knee joint. According to the present example embodiment, it is possible to perform measurements of the lower limbs based on sensor data acquired by a single sensor.

In an aspect of the present example embodiment, the detection unit detects foot adjacent and heel strike as gait events. The measurement unit converts the coordinate system of the sensor data for a prescribed period with foot adjacent as a start point into the first relative coordinate system with the position of the knee joint at the timing of foot adjacent as the origin. The measurement unit calculates the length of the lower leg and the moving speed of the knee based on the geometric model on which the first to third constraint conditions are imposed, in the first relative coordinate system. The first constraint condition is a condition that the angle formed by the lower leg and the planar surface is a right angle for the period from foot adjacent to heel strike. The second constraint condition is a condition that the extension/flexion of the knee joint is a rotational motion around the knee joint. The third constraint condition is a condition that the knee performs a constant velocity motion for a prescribed period from foot adjacent. Using the length of the lower leg and the moving speed of the knee joint, the measurement unit calculates the trajectory of the knee for the period from foot adjacent to heel strike, based on the geometric model on which the first to third constraint conditions are imposed. According to the present aspect, it is possible to measure the trajectory of the knee for the period from foot adjacent to heel strike.

In an aspect of the present example embodiment, the detection unit detects tibia vertical as a gait event. The measurement unit converts the coordinate system of the sensor data from tibia vertical to heel strike into a second relative coordinate system with the position of the knee joint at the time point of tibia vertical as the origin. The measurement unit calculates the length of the upper leg based on the geometric model on which the fourth to sixth constraint conditions are imposed, in the second relative coordinate system. The fourth constraint condition is a condition that the hip joint angle is constant for the period from tibia vertical to heel strike. The fifth constraint condition is a condition that the upper leg and the lower leg are in a straight line immediately before heel strike. The sixth constraint condition is a condition that the position of the pelvis in the sagittal plane at the timing of heel strike is the position midway between the both knees. Using the trajectory of the knee joint and the length of the upper leg, the measurement unit calculates the trajectory of the hip joint and the angle of the knee joint for the period from tibia vertical to heel strike, based on the geometric model on which the fourth to sixth constraint conditions are imposed. According to the present aspect, it is possible to calculate the trajectory of the pelvis (hip joint) and the angle of the knee joint for the period from tibia vertical to heel strike.

### Second Example Embodiment

Next, a measurement system according to a second example embodiment will be described with reference to the drawings. The measurement system of the present example embodiment generates an estimation model for estimating the physical condition of the user by learning using information on the lower limbs measured by the method of the first example embodiment.

### (Configuration)

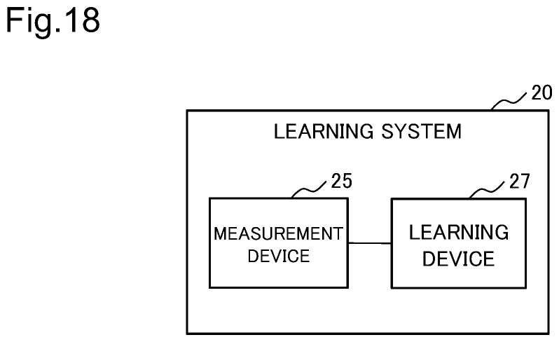

FIG. 18 is a block diagram illustrating an example of a configuration of a learning system 20 of the present example embodiment. The learning system 20 includes a measurement device 25 and a learning device 27. The measurement device 25 and the learning device 27 may be connected by wire or wirelessly. The measurement device 25 and the learning device 27 may be configured as a single device.

The measurement device 25 has the same configuration as the measurement device 15 of the first example embodiment. The measurement device 25 acquires sensor data from a data acquisition device (not illustrated). The measurement device 25 converts the coordinate system of the acquired sensor data from the local coordinate system to the world coordinate system. The measurement device 25 generates time-series data (also called a gait waveform) of the sensor data after conversion into the world coordinate system. The measurement device 25 detects gait events from the gait waveform. Based on the detected gait events, the measurement device 25 performs measurements of the lower limbs using a geometric model on which constraint conditions specific to gait are imposed. For example, the measurement device 25 measures the length of a portion between the knee joint and the ankle joint (also called lower leg) and the length of a portion between the hip joint and the knee joint (also called upper leg). For example, the measurement device 25 measures the positions of the knee joint and the hip joint. For example, the measurement device 25 measures temporal changes (trajectory) in the positions of the knee joint and the hip joint. For example, the measurement device 25 measures the angle of the knee joint.

The measurement device 25 outputs the information on the lower limbs to the learning device 27. For example, the measurement device 25 may accumulate the information on the lower limbs in a database (not illustrated). The information on the lower limbs includes foot information, knee information, and pelvis information. The foot information is information on the motion of the foot. For example, the foot information includes information such as the spatial acceleration, spatial angular velocity, spatial velocity, spatial angle (plantar angle), and spatial trajectory of the foot. The knee information is information on the motion of the knee. For example, the knee information includes information such as the position, trajectory, and angle of the knee joint. The pelvis information is information on the motion of the pelvis. For example, the pelvis information includes information such as the position and trajectory of the pelvis (hip joint).

The learning device 27 acquires information on the lower limbs from the measurement device 25. The learning device 27 may be configured to receive the information on the lower limbs accumulated in the database (not illustrated). In the case of using the information on the lower limbs accumulated in the database, the learning device 27 acquires the information on the lower limbs from the database.

The learning device 27 learns the received information on the lower limbs. For example, the learning device 27 learns the information on the lower limbs extracted from the gait waveforms of a plurality of users as teacher data. The learning device 27 generates an estimation model that has learned about a plurality of users. The learning device 27 stores the generated estimation model in a storage device (not illustrated). The estimation model learned by the learning device 27 may be stored in a storage device outside the learning device 27.

For example, the learning device 27 executes learning using a linear regression algorithm. For example, the learning device 27 executes learning using an algorithm of support vector machine (SVM). For example, the learning device 27 executes learning using an algorithm of Gaussian process regression (GPR). For example, the learning device 27 executes learning using an algorithm such as random forest (RF). For example, in accordance with input of the information on the lower limbs, the learning device 27 may execute unsupervised learning for classifying the input information. The algorithm of learning executed by the learning device 27 is not particularly limited.

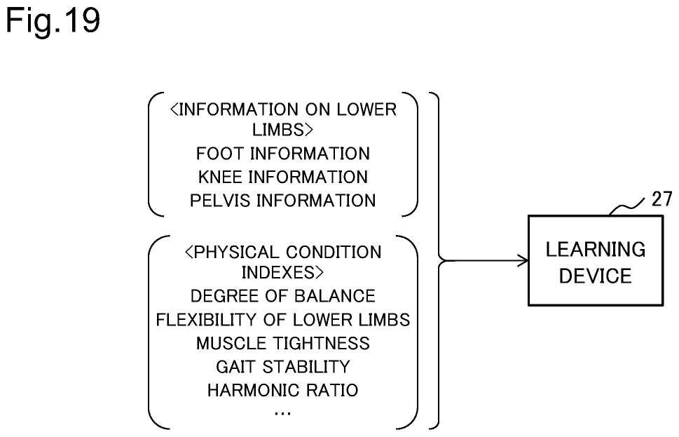

FIG. 19 is a conceptual diagram illustrating an example of causing the learning device 27 to learn a data set of information on the lower limbs which is an explanatory variable and physical condition indexes which are an objective variable as teacher data. In the example of FIG. 19, at least one of a plurality of pieces of information on the lower limbs is set as an explanatory variable, and at least one of a plurality of physical conditions is set as an objective variable. For example, the learning device 27 learns data on a plurality of subjects, and generates an estimation model that outputs index values of physical conditions in response to an input of information on the lower limbs extracted from sensor data. Hereinafter, an example of the physical condition indexes illustrated in FIG. 19 will be described. The physical condition indexes illustrated in FIG. 19 are an example, and do not limit the physical condition indexes to be learned by the learning device 27.

The degree of balance is an index of the symmetry of both legs during gait. For example, the degree of balance takes on a value obtained by quantifying differences between right and left in the information on the lower limbs during gait. The higher the symmetry of the feet during gait, the higher the degree of balance.

The flexibility of the lower limbs is an index of the range of motion of the pelvis during gait. For example, the flexibility of the lower limbs is obtained based on movement or rotation of the pelvis during gait. The greater the motion range of the pelvis during gait, the higher the flexibility of the lower limbs.

The muscle tightness is an index of muscle tonus. For example, medial rotation of the hip joint and muscle tightness of the iliopsoas, quadriceps femoris, triceps surae, and gluteal muscle groups are evaluated. As muscle tension increases, muscle tightness tends to increase.

The gait stability is an index of variation in gait. For example, the gait stability can be evaluated based on variation in acceleration of the pelvis. As the variation in gait is large, the variation in acceleration of the pelvis increases and the gait stability decreases.

The harmonic ratio is an index of symmetry of a waveform of time-series data of acceleration (also called an acceleration waveform) measured by an acceleration sensor attached to the vicinity of the pelvis or the like. In the gait motion, changes in acceleration in two cycles (one gait cycle) with one step of each of the right and left legs as one cycle are repeated. The harmonic ratio in the vertical direction (Z direction) and the traveling direction (Y direction) can be calculated as a ratio between a power sum of even harmonics corresponding to elements during the gait cycle and a power sum of odd harmonics deviating from the power sum of the even harmonics by performing Fourier transform with the time of one gait cycle as a basic cycle. Since the harmonic ratio in the left-right direction (X direction) is one cycle in two steps, the odd harmonics are regarded as an element in the gait cycle, and can be calculated as an inverse of the harmonic ratio in the vertical direction (Z direction) and the traveling direction (Y direction). The higher the harmonicity of gait, the more the acceleration change that occurs in the normal gait operation during one gait cycle is included, and the higher the harmonic ratio is. On the other hand, the harmonic ratios of a Parkinson's disease patient, a knee osteoarthritis patient, and an elderly person tend to decrease during a gait. When the harmonic ratio during a gait decreases, the risk of fall tends to increase. Therefore, the harmonic ratio is an index for the progress status of the disease and the risk of fall.

As described above, the learning system of the present example embodiment includes the measurement device and the learning device. The measurement device detects gait events from time-series data of sensor data related to the motion of a foot. The measurement device performs measurements of the lower limbs based on a geometric model on which constraint conditions related to the motion of the lower limbs are imposed, using sensor data for a prescribed period with the timing of a gait event as a start point. The learning unit learns information on the lower limbs measured by the measurement device. The learning device generates an estimation model that has learned about a plurality of subjects. The learning device stores the generated estimation model in the storage device.

The learning system according to the present example embodiment generates an estimation model that performs estimation according to information on lower limbs, through learning using information on the lower limbs measured by the measurement device. According to the present example embodiment, it is possible to generate an estimation model that performs estimation according to information on the lower limbs.

### Third Example Embodiment

Next, a measurement system according to a third example embodiment will be described with reference to the drawings. For example, the measurement system of the present example embodiment estimates the physical conditions of the user using an estimation model learned by the learning device of the second example embodiment.

### (Configuration)

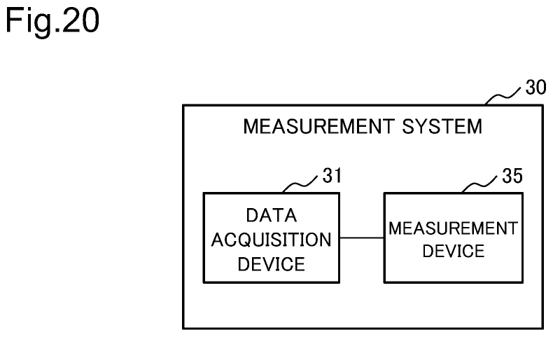

FIG. 20 is a block diagram illustrating an example of a configuration of a measurement system 30 of the present example embodiment. The measurement system 30 includes a data acquisition device 31 and a measurement device 35. The data acquisition device 31 and the measurement device 35 may be connected by wire or wirelessly. The data acquisition device 31 and the measurement device 35 may be configured as a single device. Alternatively, the measurement system 30 may be configured only by the measurement device 35, eliminating the data acquisition device 31 from the configuration of the measurement system 30. Although only one data acquisition device 31 is illustrated in FIG. 20, one each (two in total) data acquisition device 31 may be arranged on the right and left feet.

The data acquisition device 31 has the same configuration as that of the data acquisition device 11 of the first example embodiment. The data acquisition device 31 is installed on at least one of the right and left feet. The data acquisition device 31 includes an acceleration sensor and an angular velocity sensor. The data acquisition device 31 converts the measured physical quantities into digital data (also called sensor data). The data acquisition device 31 transmits the converted sensor data to the measurement device 35.

The measurement device 35 receives sensor data from the data acquisition device 31. The measurement device 35 converts the coordinate system of the acquired sensor data from the local coordinate system to the world coordinate system. The measurement device 35 generates time-series data (also called a gait waveform) of the sensor data after conversion into the world coordinate system. The measurement device 35 detects gait events from the gait waveform. As the measurement device 15 of the first example embodiment does, based on the detected gait events, the measurement device 35 performs measurements of the lower limbs using a geometric model on which constraint conditions specific to gait are imposed. The measurement device 35 estimates the physical conditions of the user based on the information on the measured lower limbs. For example, the measurement device 35 inputs information on the lower limbs to the estimation model generated by the learning device 27 of the second example embodiment, and estimates the physical conditions of the user. For example, the measurement device 35 compares information on the lower limbs measured at different timings to estimate the physical conditions of the user. The measurement device 35 outputs the estimated physical conditions. For example, the measurement device 35 outputs information on the lower limbs to a display device (not illustrated) or an external system.

### [Measurement Device]

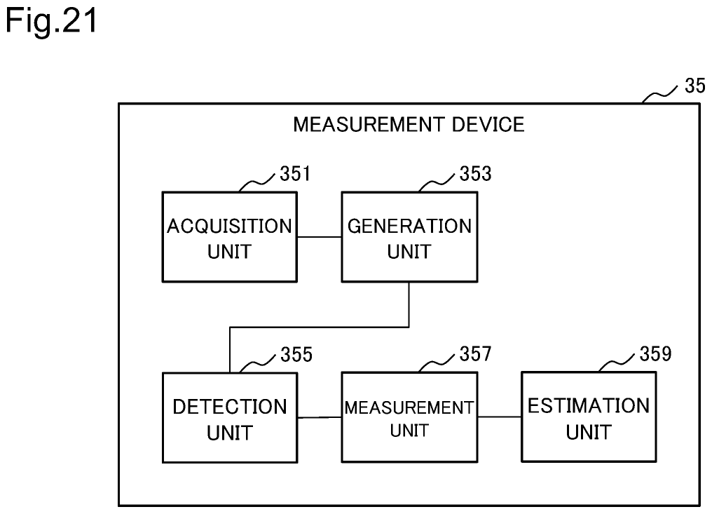

Next, details of the measurement device 35 will be described with reference to the drawings. FIG. 21 is a block diagram illustrating an example of a detailed configuration of the measurement device 35. The measurement device 35 includes an acquisition unit 351, a generation unit 353, a detection unit 355, a measurement unit 357, and an estimation unit 359.

The acquisition unit 351 has the same configuration as the acquisition unit 151 of the first example embodiment. The acquisition unit 351 receives sensor data from the data acquisition device 31. The acquisition unit 351 outputs the received sensor data to the generation unit 353.

The generation unit 353 has the same configuration as the generation unit 153 of the first example embodiment. The generation unit 353 acquires sensor data from the acquisition unit 351. The generation unit 353 converts the coordinate system of the acquired sensor data from the local coordinate system to the world coordinate system. The generation unit 353 generates time-series data (also called a gait waveform) of the sensor data after conversion into the world coordinate system. The generation unit 353 outputs the generated gait waveform to the detection unit 355.

The detection unit 355 has the same configuration as the detection unit 155 of the first example embodiment. The detection unit 355 acquires the gait waveform from the generation unit 353. The detection unit 355 detects gait events from the gait waveform. The detection unit 355 outputs the timings of the detected gait events and the values of sensor data for a prescribed period with a gait event as a start point to the measurement unit 357.

The measurement unit 357 has the same configuration as the measurement unit 157 of the first example embodiment. The measurement unit 357 acquires, from the detection unit 355, the timing of a gait event and the value of the sensor data for a prescribed period with the timing of the gait event as a start point. The measurement unit 357 applies the acquired values of the sensor data to a geometric model on which constraint conditions are imposed, and performs measurements of the lower limbs.

The measurement unit 357 outputs the information on the measured motion of the lower limbs to the estimation unit 359. For example, the information on the lower limbs includes foot information, knee information, and pelvis information. The foot information is information on the motion of the foot. For example, the foot information includes information such as the spatial acceleration, spatial angular velocity, spatial velocity, spatial angle (plantar angle), and spatial trajectory of the foot. The knee information is information on the motion of the knee. For example, the knee information includes information such as the position, trajectory, and angle of the knee joint. The pelvis information is information on the motion of the pelvis. For example, the pelvis information includes information such as the position and trajectory of the pelvis (hip joint).

The estimation unit 359 acquires information on the lower limbs from the measurement unit 357. The estimation unit 359 estimates the physical conditions of the user using the acquired information on the lower limbs. For example, the estimation unit 359 estimates the physical conditions of the user based on index values output in response to an input of information on the lower limbs such as foot information, knee information, and pelvis information to the estimation model. For example, the estimation unit 359 compares a plurality of pieces of information on the lower limbs measured with different timings to estimate the physical conditions of the user.

The estimation unit 359 outputs estimation results based on the information on the lower limbs. For example, in the case of using an estimation model stored in a storage device such as an external server, the estimation model may be used via an interface (not illustrated) connected to the storage device. For example, the estimation unit 359 outputs the estimation results of the physical conditions to a display device (not illustrated). The estimation results of the physical conditions output to the display device are displayed on the screen of the display device. For example, the estimation unit 359 outputs the estimation results of the physical conditions to an external system. The estimation results of the physical conditions output to the external system are used for any purpose.

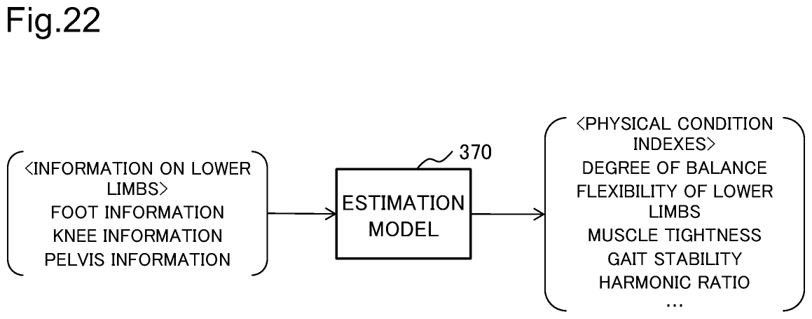

FIG. 22 is a conceptual diagram illustrating an example in which the index values of the physical conditions of the user are output in response to an input of information on the lower limbs measured along with gait of the user to an estimation model 370 constructed in advance. The physical conditions corresponding to the input information on the lower limbs are output from the estimation model 370. In the example of FIG. 22, at least one of the plurality of pieces of information on the lower limbs is input to the estimation model 370, and at least one of the plurality of physical conditions is output from the estimation model 370. As long as the estimation results of the physical conditions can be output in response to an input of the information on the lower limbs, the results of estimation using the estimation model 370 are not limited.

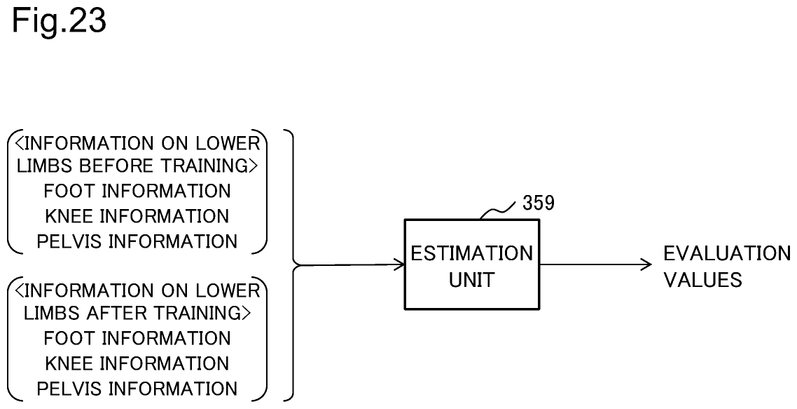

FIG. 23 is a conceptual diagram illustrating an example in which the evaluation values according to changes in the information on the lower limbs is output in response to an input of the information on the lower limbs measured with different timings along with the gait of the user. Information on the lower limbs measured with different timings is input to the estimation unit 359. The estimation unit 359 outputs evaluation values corresponding to changes in the information on the lower limbs measured with different timings. In the example of FIG. 23, the information on the lower limbs before and after the training is input to the estimation unit 359. In the example of FIG. 23, evaluation values corresponding to changes in the information on the lower limbs before and after the training are output. For example, if the information on the lower limbs indicates improvement after the training as compared with before the training, the estimation unit 359 outputs evaluation values indicating that the effect of the training was good. For example, if the information on the lower limbs indicates deterioration after the training as compared with before the training, the estimation unit 359 outputs evaluation values indicating that the effect of the training was not good. There is no limitation on the estimation results from the estimation unit 359 as long as the estimation results (evaluation values) regarding the changes in the physical conditions can be output in response to an input of the information on the lower limbs measured with different timings.

### APPLICATION EXAMPLES

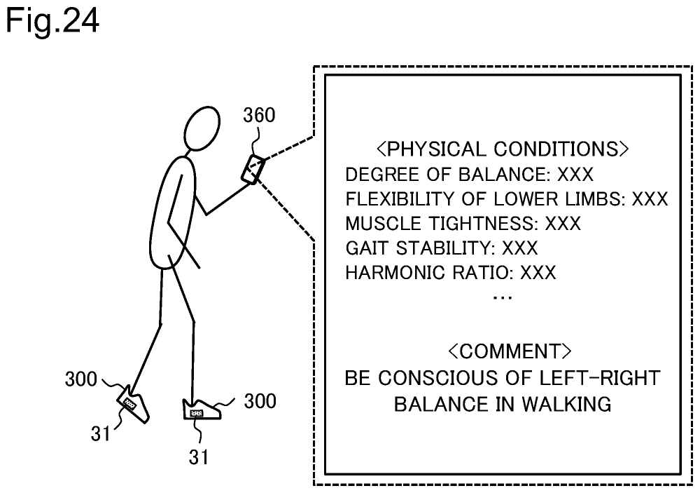

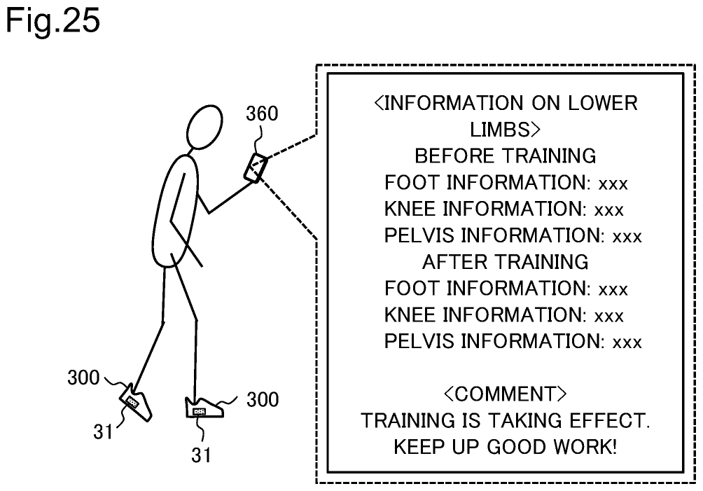

Application examples of the present example embodiment will be described with reference to the drawings. FIGS. 24 and 25 are conceptual diagrams for describing examples of application of the present example embodiment. In the following application examples, sensor data is transmitted to a mobile terminal 360 carried by a user according to gait of the user wearing shoes 300 in which the data acquisition devices 31 are installed. The app (measurement device 35) installed in the mobile terminal 360 displays information on the physical conditions of the user on the screen of the mobile terminal 360 based on the received sensor data.

### Application Example 1

FIG. 24 is a conceptual diagram for describing Application Example 1. In the present application example, the physical conditions are estimated based on the information on the lower limbs using the estimation model 370 generated by the method in FIG. 22. It is assumed that an app having the function of the measurement device 35 is installed in the mobile terminal 360.

For example, the app generates recommendation information in accordance with the estimated index values of the physical conditions. For example, when the index value of a certain physical condition exceeds a threshold value, the app generates recommendation information on which the index value may be reduced. For example, when the index value of a certain physical condition falls below a threshold value, the app generates recommendation information on which the index value may be increased. For example, if the index value of a certain physical state is close to a threshold value, the app generates recommendation information for recommending the user to maintain the gait state at that time. For example, the app displays recommendation information in accordance with the estimated physical conditions on the screen of the mobile terminal 360.

In the example of FIG. 24, in response to lack in the left-right balance in gait, recommendation information “Be conscious of left-right balance in walking” is displayed on the screen of the mobile terminal 360. For example, the user who has seen the information displayed on the screen of the mobile terminal 360 can recognize his/her physical conditions according to the information.

In the present application example, the recommendation information according to the physical conditions estimated based on the information on lower limbs is displayed on a screen of the mobile terminal 360 carried by the user. Therefore, according to the present application example, the recommendation information reflecting the physical conditions of the user can be provided to the user via the screen of the mobile terminal 360. For example, for a user who has a pain in the waist due to lack in the left-right balance in gait, it is desirable to walk in such a way that the left-right balance is maintained normally. According to the present application example, since it is recommended to be conscious of the left-right balance in gait, the user can continue walking while maintaining an appropriate balance.

### Application Example 2

FIG. 25 is a conceptual diagram for describing Application Example 2 of the present example embodiment. In the present application example, the physical conditions are estimated by using the method in FIG. 23 to compare a plurality of pieces of information on the lower limbs measured with different timings. It is assumed that an app having the function of the measurement device 35 is installed in the mobile terminal 360.

For example, the app generates notification information in accordance with the estimated evaluation values. For example, if the evaluation values of the information on the lower limbs exceed the target values, the app generates notification information indicating that the targets have been achieved. For example, if any of the evaluation values of the information on the lower limbs does not exceed the target value, the app generates notification information indicating that the target has not been achieved. For example, the app causes the notification information corresponding to the estimated evaluation values to be displayed on the screen of the mobile terminal 360.

In the example of FIG. 25, in response to the evaluation values of the information on the lower limbs such as the trajectories of the knee and the pelvis exceeding the target values after the training, notification information “Training is taking effect. Keep up good work” is displayed on the screen of the mobile terminal 360. For example, on seeing the information displayed on the screen of the mobile terminal 360, the user can recognize the effect of the training according to the information.

In the present application example, the notification information corresponding to the evaluation values estimated based on the information on the lower limbs measured with different timings is displayed on the screen of the mobile terminal 360 carried by the user. Therefore, according to the present application example, it is possible to provide the user with the notification information according to changes in the information on the lower limbs measured with different timings. For example, for a user who has a pain in the waist due to lack in the left-right balance in gait, it is desirable to walk in such a way that the left-right balance is maintained normally. According to the present application example, since the training effect according to the change in the left-right balance in gait, the user can continue the appropriate training.

As described above, the measurement system of the present example embodiment includes the data acquisition device and the measurement device. The data acquisition device is arranged on a user's footwear. The data acquisition device measures a spatial acceleration and a spatial angular velocity according to the gait of the user. The data acquisition device generates sensor data based on the measured spatial acceleration and spatial angular velocity. The data acquisition device outputs the generated sensor data to the measurement device. The measurement device includes the acquisition unit, the generation unit, the detection unit, the measurement unit, and the estimation unit. The acquisition unit acquires sensor data related to the motion of the foot. The generation unit generates time-series data of sensor data related to the motion of the foot. The detection unit detects gait events from the time-series data of sensor data related to the motion of the foot. The measurement unit performs measurements of the lower limbs based on a geometric model on which constraint conditions related to the motion of the lower limbs are imposed, using sensor data for a prescribed period with the timing of a gait event as a start point. The estimation unit estimates the physical conditions of the user based on the information on the lower limbs of the user.

The measurement device of the present example embodiment can estimate the physical conditions of the user based on the information on the lower limbs measured using the time-series data of the sensor data.

In an aspect of the present example embodiment, the estimation unit inputs the information on the lower limbs of the user to the estimation model that outputs the index values of the physical conditions in response to an input of the information on the lower limbs. The estimation unit estimates the physical conditions of the user based on the index values output from the estimation model in response to the input of the information on the lower limbs. The estimation unit outputs recommendation information according to the physical conditions of the user. According to this aspect, it is possible to provide the recommendation information according to the physical conditions of the user based on the information on the lower limbs of the user using an estimation model generated in advance.

In an aspect of the present example embodiment, the estimation unit compares a plurality of pieces of information on the lower limbs measured with different timings. The estimation unit estimates the physical conditions of the user based on the evaluation values of the comparison results of the plurality of pieces of information on the lower limbs. The estimation unit outputs notification information according to the physical conditions of the user. According to this aspect, it is possible to provide the notification information according to the physical conditions of the user based on the information on the lower limbs of the user using an estimation model generated in advance.

In an aspect of the present example embodiment, the estimation unit outputs information according to the physical conditions of the user to the terminal device carried by the user. According to the present aspect, the user can see the information displayed on the display unit of the terminal device to recognize his/her body information.

### Fourth Example Embodiment

Next, a measurement device according to a fourth example embodiment will be described with reference to the drawings. The measurement device of the present example embodiment has a configuration in which the measurement devices of the first to third example embodiments are simplified.

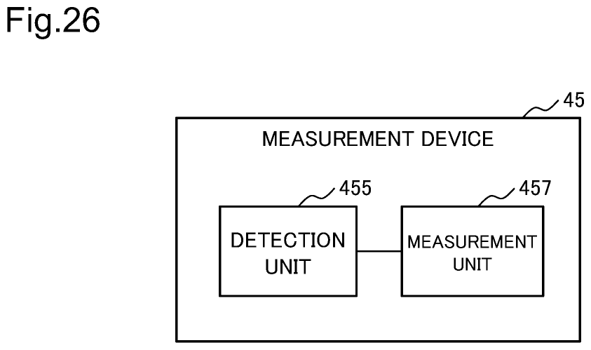

FIG. 26 is a block diagram illustrating an example of a configuration of a measurement device 45 of the present example embodiment. The measurement device 45 includes a detection unit 455 and a measurement unit 457. The detection unit 455 detects gait events from the time-series data of sensor data related to the motion of the foot. The measurement unit 457 performs measurements of the lower limbs based on a geometric model on which constraint conditions related to the motion of the lower limbs are imposed, using sensor data for a prescribed period with the timing of a gait event as a start point.

The measurement device of the present example embodiment performs measurements of the lower limbs based on knowledge of biomechanics using time-series data of sensor data acquired by a single sensor. That is, according to the present example embodiment, it is possible to perform measurements of the lower limbs based on sensor data acquired by a single sensor.

### (Hardware)

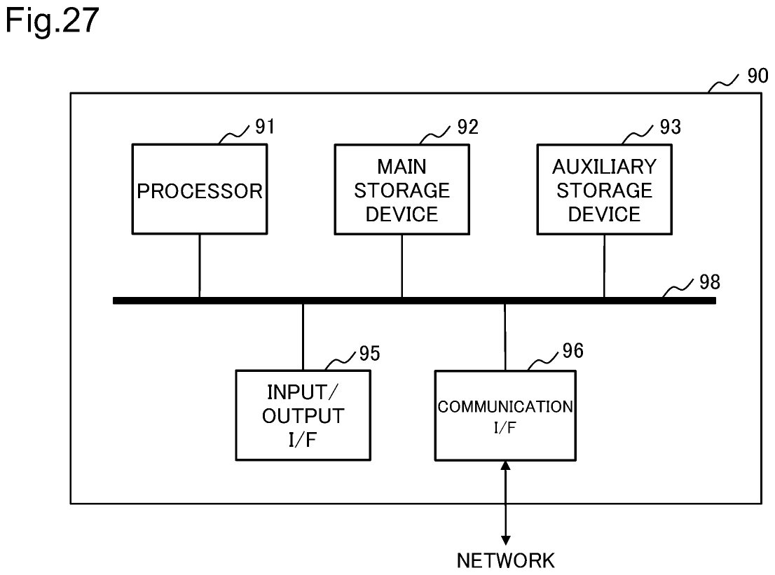

Here, a hardware configuration for executing control and processing according to each example embodiment of the present disclosure will be described taking an information processing apparatus 90 in FIG. 27 as an example. The information processing apparatus 90 in FIG. 27 is taken for describing a configuration example for executing control and processing of each example embodiment, and does not limit the scope of the present disclosure.

As illustrated in FIG. 27, the information processing apparatus 90 includes a processor 91, a main storage device 92, an auxiliary storage device 93, an input/output interface 95, and a communication interface 96. In FIG. 27, the interface is abbreviated as an interface (I/F). The processor 91, the main storage device 92, the auxiliary storage device 93, the input/output interface 95, and the communication interface 96 are connected to each other in a data-communicable manner via a bus 98. The processor 91, the main storage device 92, the auxiliary storage device 93, and the input/output interface 95 are connected to a network such as the Internet or an intranet via the communication interface 96.

The processor 91 develops the program stored in the auxiliary storage device 93 or the like, in the main storage device 92. The processor 91 executes the program developed in the main storage device 92. In the present example embodiment, a software program installed in the information processing apparatus 90 may be used. The processor 91 executes control and processing according to the present example embodiment.

The main storage device 92 has an area in which a program is developed. Programs stored in the auxiliary storage device 93 or the like are developed in the main storage device 92 by the processor 91. The main storage device 92 is implemented by a volatile memory such as a dynamic random access memory (DRAM), for example. A nonvolatile memory such as a magnetoresistive random access memory (MRAM) may be configured/added as the main storage device 92.

The auxiliary storage device 93 stores various types of data such as programs. The auxiliary storage device 93 is implemented by a local disk such as a hard disk or a flash memory. The various types of data may be stored in the main storage device 92, and the auxiliary storage device 93 may be omitted.

The input/output interface 95 is an interface for connecting the information processing apparatus 90 and a peripheral device based on a standard or a specification. The communication interface 96 is an interface for connecting to an external system or device through a network such as the Internet or an intranet based on a standard or a specification. The input/output interface 95 and the communication interface 96 may be unified as an interface connected to an external device.

An input device such as a keyboard, a mouse, or a touch panel may be connected to the information processing apparatus 90 as necessary. These input devices are used to input information and settings. When the touch panel is used as an input device, the display screen of the display device may also serve as the interface of the input device. Data communication between the processor 91 and the input device may be mediated by the input/output interface 95.

The information processing apparatus 90 may be provided with a display device for displaying information. When a display device is provided, the information processing apparatus 90 preferably includes a display control device (not illustrated) for controlling display of the display device. The display device may be connected to the information processing apparatus 90 via the input/output interface 95.

The information processing apparatus 90 may be provided with a drive device. The drive device mediates reading of data and programs from a recording medium (program recording medium), writing of a processing result of the information processing apparatus 90 to the recording medium, and the like, between the processor 91 and the recording medium. The drive device may be connected to the information processing apparatus 90 via the input/output interface 95.

The above is an example of a hardware configuration for enabling control and processing according to each example embodiment of the present invention. The hardware configuration in FIG. 27 is an example of a hardware configuration for executing control and processing according to each example embodiment, and does not limit the scope of the present invention. A program for causing a computer to execute control and processing according to each example embodiment is also included in the scope of the present invention. Further, a program recording medium in which the program according to each example embodiment is recorded is also included in the scope of the present invention. The recording medium can be implemented by an optical recording medium such as a compact disc (CD) or a digital versatile disc (DVD), for example. The recording medium may be implemented by a semiconductor recording medium such as a universal serial bus (USB) memory or a secure digital (SD) card. The recording medium may be implemented by a magnetic recording medium such as a flexible disk, or another recording medium. When a program executed by the processor is recorded in a recording medium, the recording medium corresponds to a program recording medium.

The components of each example embodiment may be arbitrarily combined. The components of each example embodiment may be implemented by software or may be implemented by a circuit.

While the invention has been particularly shown and described with reference to exemplary embodiments thereof, the invention is not limited to these embodiments. It will be understood by those of ordinary skill in the art that various changes in form and details may be made therein without departing from the spirit and scope of the present invention as defined by the claims.

This application is based upon and claims the benefit of priority from Japanese patent application No. 2021-067830, filed on Apr. 13, 2021, the disclosure of which is incorporated herein in its entirety by reference.

### REFERENCE SIGNS LIST

10, 30 Measurement system
11, 31 Data acquisition device
15, 25, 35, 45 Measurement device
20 Learning system
27 Learning device
111 Acceleration sensor
112 Angular velocity sensor
113 Control unit
115 Transmission unit
151, 351 Acquisition unit
153, 353 Generation unit
155, 355, 455 Detection unit
157, 357, 457 Measurement unit
359 Estimation unit

What is claimed is:

1. A measurement device comprising:
a memory storing instructions; and
a processor connected to the memory and configured to execute the instructions to:
detect a gait event from time-series data of sensor data related to motion of a foot; and
perform a measurement of lower limbs by using the sensor data for a prescribed period with a timing of the gait event as a start point, based on a geometric model on which a constraint condition related to motion of the lower limbs is imposed.

2. The measurement device according to claim 1, wherein
the processor is configured to execute the instructions to detect a foot adjacent and a heel strike as the gait event,
convert a coordinate system of the sensor data for a prescribed period with foot adjacent as a start point into a first relative coordinate system with a position of a knee joint at a timing of the foot adjacent as an origin,
calculate a length of a lower leg and a moving speed of a knee, based on the geometric model on which a first constraint condition that an angle formed by the lower leg and a planar surface is a right angle for a period from the foot adjacent to the heel strike, a second constraint condition that extension/bending of the knee joint is a rotational motion around the knee joint, and a third constraint condition that a knee performs a constant velocity motion for the prescribed period are imposed in the first relative coordinate system, and
calculate a trajectory of the knee for the period from the foot adjacent to the heel strike, using the length of the lower leg and the moving speed of the knee, based on the geometric model on which the first constraint condition, the second constraint condition, and the third constraint condition are imposed.

3. The measurement device according to claim 2, wherein
the processor is configured to execute the instructions to detect tibia vertical as the gait event,
convert a coordinate system of the sensor data from the tibia vertical to the heel strike into a second relative coordinate system with the position of the knee joint at a time point of the tibia vertical as an origin,
calculate a length of an upper leg, based on the geometric model on which a fourth constraint condition that an angle of a hip joint is constant for the period from the tibia vertical to the heel strike, a fifth constraint condition that the upper leg and the lower leg are in a straight line immediately before the heel strike, and a sixth constraint condition that a position of a pelvis in a sagittal plane at the timing of the heel strike is a position midway between both knees are imposed in the second relative coordinate system, and
calculate a trajectory of the hip joint and an angle of the knee joint for the period from the tibia vertical to the heel strike, using a trajectory of the knee joint and the length of the upper leg, based on the geometric model on which the fourth constraint condition, the fifth constraint condition, and the sixth constraint condition are imposed.

4. The measurement device according to claim 1, wherein
the processor is configured to execute the instructions to
estimate a physical condition of the user based on information on the lower limbs of the user.

5. The measurement device according to claim 4, wherein
the processor is configured to execute the instructions to input the information on the lower limbs of the user to an estimation model that outputs an index value of the physical condition in response to an input of the information on the lower limbs,
estimate the physical condition of the user based on the index value output from the estimation model in response to the input of the information on the lower limbs, and
output recommendation information according to the physical condition of the user.

6. The measurement device according to claim 4, wherein
the processor is configured to execute the instructions to
compare a plurality of pieces of information on the lower limbs measured with different timings,
estimate the physical condition of the user based on an evaluation value related to a comparison result of the plurality of pieces of information on the lower limbs, and
output notification information according to the physical condition of the user.

7. The measurement device according to claim 5, wherein
the processor is configured to execute the instructions to output the information according to the physical condition of the user to a terminal device carried by the user.

8. An information processing system comprising:
the measurement device according to claim 1; and
a data acquisition device that is arranged on footwear of a user, measures a spatial acceleration and a spatial angular velocity according to a gait of the user, generates sensor data based on the measured spatial acceleration and spatial angular velocity, and outputs the generated sensor data to the measurement device.

9. A measurement method comprising causing a computer to execute:
detecting a gait event from time-series data of sensor data related to motion of a foot; and
performing a measurement of lower limbs by using the sensor data for a prescribed period with a timing of the gait event as a start point, based on a geometric model on which a constraint condition related to motion of the lower limbs is imposed.

10. A non-transitory recording medium recording a program for cause a computer to execute:
detecting a gait event from time-series data of sensor data related to motion of a foot; and
performing a measurement of lower limbs by using the sensor data for a prescribed period with a timing of the gait event as a start point, based on a geometric model on which a constraint condition related to motion of the lower limbs is imposed.

11. A machine learning system comprising:
the measurement device according to claim 1; and
a learning device comprising
a memory storing instructions, and
a processor connected to the memory and configured to execute the instructions to
acquire data related to lower limbs,
generate an estimation model that outputs an index value of a physical condition in response to an input of information on the lower limbs by performing a machine learning, and
generate recommendation information for making decision related to health of a user in response to the index value estimated by using the estimation model.
